SEQUENCE LISTING (14 sequences)

SEQ ID NO: 1 - protein, 425 aa, synthetic construct
MASGQFVNKLQEEVICPICLDILQKPVTIDCGHNFCLKCITQIGETSCGFFKCPLCKTSVRKNAIRFNSLLRNLVEKIQALQASEVQSKRKEATCPRHQEMFHYFCEDDGKFLCFVCRESKDHKSHNVSLIEEAAQNYQGQIQEQIQVLQQKEKETVQVKAQGVHRVDVFTDQVEHEKQRILTEFELLHQVLEEEKNFLLSRIYWLGHEGTEAGKHYVASTEPQLNDLKKLVDSLKTKQNMPPRQLLEDIKVVLCRSEEFQFLNPTPVPLELEKKLSEAKSRHDSITGSLKKFKDQLQADRKKDENRFFKSMNKNDMKSWGLLQKNNHKMNKTSEPGSSSAGGRTTSGPPNHHSSAPSHSLFRASSAGKVTFPVCLLASYDEISGQGASSQDTKTFDVALSEELHAALSEWLTAIRAWFCEVPSS

SEQ ID NO: 2 - protein, 507 aa, synthetic construct
MAGQPLACQLQEEVTCPICMEILQDPVTIDCGHNFCLQCISQVGKTSEKIQCPLCKLSVNKNTFRPNKLLASLAEKIQSMDPADIQAEKEDSRCQRHKEKLHYFCEQDGAFLCVVCRDSKDHKSHNVTLIDEAAQNYKVQIESQAQDLGQKDKKIIEEKKQGEGAIWAFRAQVDLEKLKIHEEFKLLRQRLDEEESFLLSRLDWLEQQGAKQLRQYVTVTEKQLNSLRKLTKSLKIRLQSSSMELLKDIKDALSRGKEFQFLNPNPVPEDLEKKCSEAKARHESIIKTLTELKDDMNAEGKRDKSAFMNSLNKEEKESWSLLQKNNSVLPTSVPVTLDKSSADPDLTFSQDLKKVTLYIVAGKASNRQAKPRPFYPFHCVRGSPGLSSGRQVWEAEIRGPSGGACIVGVVTELARGAQSQTVSAQSYIWALRISPSGCQPFTNCKAQEYLQVCLKKVGVYVNHDCGEVVFYDAITSKHIYTFQTSFDGKVFPLFGLQVACSHITLSP

SEQ ID NO: 3 - protein, 856 aa, synthetic construct
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 4 - protein, 827 aa, synthetic construct
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 5 - DNA, 2027 bp, synthetic construct
  source 1..2027: mol_type = other DNA
atccatttgttctcaagaggtagcaagaaacagtatccacagtggactccggggctcctacagacttggcacagcttcctacagtcttgaaacagccctgttgttctgtcatggccagtgggcagtttgtgaacaaactgcaagaggaagtgatctgccccatctgcctggacattctgcagaaacctgtcaccatcgactgtgggcacaatttctgcctcaaatgcatcactcagattggggaaacatcatgtggatttttcaaatgtcccctctgcaaaacttccgtaaggaagaacgcaatcaggttcaactcgctgttgcggaatctggtggagaaaatccaagctctacaagcctctgaggtgcagtccaaaaggaaagaggctacatgcccgaggcaccaggagatgttccactatttctgcgaggatgatgggaagttcctctgttttgtgtgtcgtgaatccaaggaccacaaatcccataatgtcagcttgatcgaagaagctgcccagaattatcaggggcagattcaagagcagatccaagtcttgcagcaaaaggagaaggagacagtacaagtgaaggcacaaggtgtacacagggtcgatgtcttcacggaccaggtagaacatgagaagcaaaggatcctcacagaatttgaactcctgcatcaagtcctagaggaggagaagaatttcctgctatcacggatttactggctgggtcatgagggaacggaagcggggaaacactatgttgcctccactgagccacagttgaacgatctcaagaagctcgttgattccctgaagaccaagcagaacatgccacccaggcagctgctggaggatatcaaagtcgtcttgtgcagaagtgaagagtttcagtttctcaacccaacccctgttcctctggaactggagaaaaaactcagtgaagcaaaatcaagacatgactccatcacagggagcctaaaaaaattcaaagaccaactccaggctgataggaaaaaagatgaaaacagattcttcaaaagcatgaataaaaatgacatgaagagctggggcttgttacagaaaaataatcataaaatgaacaaaacctcagagcccgggtcatcttctgcaggcggcagaactacatcggggccaccaaatcaccactcttcagccccatcccactccctgtttcgggcctcgtctgctgggaaagtcacttttccagtatgtctcctggcctcttatgatgagatttctggtcaaggagcgagctctcaggatacgaagacatttgacgttgcgctgtccgaggagctccatgcggcactgagtgagtggctgacagcgatccgggcttggttttgtgaggttccttcaagctaagccagctcagagaacacggggagcggtggtgctacacggacttcggagcatagagtggcgctgagtgagtggctgagaccgaccacggttcttgacttagtggaattgggtcgaaggagtggagaatgggagggctcgggctactgagagtggagatgggggcgggggtggtggtgaagagagttggagaaggaatggacgaattcttgagcaaaaggaggggaagagacaatctccagccacccgccccacgcttgacttcttatcactttggctgtggtgccgcctagtggaaaaaggaagtccctgcagcagtccccgcactctttaagcagctgtttaccgaaggcaccagttcagccaggagtgaaatccggagaggagcaacgccagcctgggtcacagtccatcaaaccccatgagcccgaccactctcgctcttccttacattcccacgtcccccttctctcccaacccctcatatcagcaagggaaattaattaatgagatttgataaatcagtagatagaatgaggtccccattctgaaatatttagcagactggaaccaccacgcaagcctctgtagggggtggatggagacacttctaactttaataaactgcgactgaacgtggaa

SEQ ID NO: 6 - DNA, 2248 bp, synthetic construct
  source 1..2248: mol_type = other DNA
cagggagagaggactgaccatctgttttcttctctttagaatattccccatttatcgctgactcttttctgtcattatttatttaacccccccttccctcattctcctctaatcctctccctgagatactcttcaacctctgcctcctgcttccttctctcttacaagcaccaggccttggatttctgtactttcacatctcagattagccctgctgttccatcatggcaggccaacctctggcctgccaactgcaggaggaggtgacctgccccatctgcatggaaattctacaagatcctgtcaccattgactgtgggcacaacttctgcctgcagtgtatcagtcaggttggaaaaacgtcagaaaagatccaatgtcctctctgcaaactctctgtgaataagaatacgttcaggcccaataagctgctggctagtcttgcagagaagatccaaagtatggatcctgctgacatccaagcagaaaaggaagactcaagatgtcagaggcacaaggaaaagctacattacttctgcgagcaggatggggcattcctctgtgtggtgtgtcgtgactccaaggaccacaagtctcacaatgtcaccttgatagatgaggctgcccagaactacaaggtgcagattgagtcacaggcccaagatttggggcaaaaggacaagaaaataattgaagagaaaaaacaaggtgaaggggcaatctgggcgttcagggcccaggtggatctagagaaactaaagatccatgaggaattcaagctcctgcgccagagactagatgaggaagagagtttcctcctgtccaggctggactggctggagcagcagggagccaagcagttgagacaatatgtcactgtgacagagaagcagctgaactctctaaggaagctcactaaatccttgaaaataaggctgcaatcatcatccatggagctactaaaggacattaaagacgccttgagcaggggtaaggaatttcagtttctcaacccaaacccagttcctgaggacctggagaaaaaatgcagtgaagcaaaagcaaggcatgagtccatcataaaaaccctgacagaactcaaagacgatatgaatgccgaagggaagagagataaaagcgcattcatgaacagcctgaataaagaagaaaaggagagctggagcctgttacagaagaataattcagtgttgcccacctcagtccctgtgaccctggacaagtcctcagctgacccagacctcaccttttctcaggatctaaagaaagtgaccttgtatattgtagctggaaaagcttccaataggcaggcaaaacctcgaccattctaccctttccactgtgtgaggggttccccaggcctctcctcaggccgccaggtgtgggaggcagaaatccgggggccctcaggtggggcatgcattgtgggcgtggttacagagttggcgcgaggggcccagagtcaaaccgtgagcgcgcagagctacatatgggcactgcggatctcgccctctggatgccagccattcaccaattgtaaagcccaggagtacctccaggtctgtcttaagaaagtgggtgtctatgtgaatcatgactgtggagaggtcgtcttctatgatgccatcactagcaaacacatctatactttccaaacttcctttgacgggaaggtcttcccccttttcgggctccaagttgcctgcagccatatcaccctgagcccttagggctgctcgcttgtgtgttcctcagtctcttcccacctatcactcaggacgggatgagacaaatggtagaagtgaagtgtggccctactctgacactctacatgccacacaatcacctgatgtttcctctccctgtctcccccaagtagagaagacagtggaataccccccaaagactatacacgaggtttttcagtgctgcacttggtagcaggacctcagaaaagcttctggctgcaccctgctgacacaggggtgcaaggcgctgtccaacacattggctgctgctttctctactgtattacctgaaactcttgtgactcatcctttcctgagatctgagaaccagaggttgtacagagtcggccagttctgtgccaatgccccgtttgtagatttggacaatctacaatcaagaataaaaccttttgacatttcaaagcaactttgtcatcctcaacagctctgtcttctaattaaaagcaaaattgtaaaccacc

SEQ ID NO: 7 - DNA, 3511 bp, synthetic construct
  source 1..3511: mol_type = other DNA
gaggagccaccgcccagccgctcgcaggcgccgcacggagttgcgtcccggggacttggggccgcagggagctgtgagtacccaggaagctgcaccgtgtggcctggagctgtctatctgtccttccagccacctgtctgtccagccacccttccacagactgaggcttgacaccggagcatctgtacagagcaaggagaagacaagaacatgctctaaagcccttcacagcaagacccaggaagccgcgggcaaactcagactcgaagccctcccgcctcctgcccacaatggcctctgctgacaagaatggcgggagcgtgtcctctgtgtccagcagccgcctgcagagccggaagccacccaacctctccatcaccatcccgccacccgagaaagagacccaggcccctggcgagcaggacagcatgctgcctgagaggaagaacccagcctacttgaagagcgtcagcctccaggagccacgcagccgatggcaggagagttcagagaagcgccctggcttccgccgccaggcctcactgtcccagagcatccgcaagggcgcagcccagtggtttggagtcagcggcgactgggaggggcagcggcagcagtggcagcgccgcagcctgcaccactgcagcatgcgctacggccgcctgaaggcctcgtgccagcgtgacctggagctccccagccaggaggcaccgtccttccagggcactgagtccccaaagccctgcaagatgcccaagattgtggatccgctggcccggggccgggccttccgccacccggaggagatggacaggccccacgccccgcacccaccgctgacccccggagtcctgtccctcacctccttcaccagtgtccgttctggctactcccacctgccacgccgcaagagaatgtctgtggcccacatgagcttgcaagctgccgctgccctcctcaaggggcgctcggtgctggatgccaccggacagcggtgccgggtggtcaagcgcagctttgccttcccgagcttcctggaggaggatgtggtcgatggggcagacacgtttgactcctccttttttagtaaggaagaaatgagctccatgcctgatgatgtctttgagtcccccccactctctgccagctacttccgagggatcccacactcagcctcccctgtctcccccgatggggtgcaaatccctctgaaggagtatggccgagccccagtccccgggccccggcgcggcaagcgcatcgcctccaaggtgaagcactttgcctttgatcggaagaagcggcactacggcctcggcgtggtgggcaactggctgaaccgcagctaccgccgcagcatcagcagcactgtgcagcggcagctggagagcttcgacagccaccggccctacttcacctactggctgaccttcgtccatgtcatcatcacgctgctggtgatttgcacgtatggcatcgcacccgtgggctttgcccagcacgtcaccacccagctggtgctgcggaacaaaggtgtgtacgagagcgtgaagtacatccagcaggagaacttctgggttggccccagctcgattgacctgatccacctgggggccaagttctcaccctgcatccggaaggacgggcagatcgagcagctggtgctgcgcgagcgagacctggagcgggactcaggctgctgtgtccagaatgaccactccggatgcatccagacccagcggaaggactgctcggagactttggccacttttgtcaagtggcaggatgacactgggccccccatggacaagtctgatctgggccagaagcggacttcgggggctgtctgccaccaggaccccaggacctgcgaggagccagcctccagcggtgcccacatctggcccgatgacatcactaagtggccgatctgcacagagcaggccaggagcaaccacacaggcttcctgcacatggactgcgagatcaagggccgcccctgctgcatcggcaccaagggcagctgtgagatcaccacccgggaatactgtgagttcatgcacggctatttccatgaggaagcaacactctgctcccaggtgcactgcttggacaaggtgtgtgggctgctgcccttcctcaaccctgaggtcccagatcagttctacaggctctggctgtctctcttcctacatgctggcgtggtgcactgcctcgtgtctgtggtctttcaaatgaccatcctgagggacctggagaagctggccggctggcaccgtatcgccatcatcttcatcctcagtggcatcacaggcaacctcgccagtgccatctttctcccataccgggcagaggtgggcccggccggctcacagttcggcctcctcgcctgcctcttcgtggagctcttccagagctggccgctgctggagaggccctggaaggccttcctcaacctctcggccatcgtgctcttcctgttcatctgtggcctcctgccctggatcgacaacatcgcccacatcttcggcttcctcagtggcctgctgctggccttcgccttcctgccctacatcaccttcggcaccagcgacaagtaccgcaagcgggcactcatcctggtgtcactgctggcctttgccggcctcttcgccgccctcgtgctgtggctgtacatctaccccattaactggccctggatcgagcacctcacctgcttccccttcaccagccgcttctgcgagaagtatgagctggaccaggtgctgcactgaccgctgggccacacggctgcccctcagccctgctggaacagggtctgcctgcgagggctgccctctgcagagcgctctctgtgtgccagagagccagagacccaagacagggcccgggctctggacctgggtgcccccctgccaggcgaggctgactccgcgtgagatggttggttaaggcggggtttttctggggcgtgaggcctgtgagatcctgacccaagctcaggcacacccaaggcacctgcctctctgagtcttgggtctcagttcctaatatcccgctccttgctgagaccatctcctggggcagggtccttttcttcccaggtcctcagcgctgcctctgctggtgccttctcccccactactactggagcgtgcccttgctggggacgtggctgtgccctcagttgcccccagggctgggtgcccaccatgccccttcctctttctcctcctacctctgccctgtgagcccatccataaggctctcagatgggacattgtgggaaaggctttggccatggtctgggggcagagaacaaggggggagacacaagtagacctcaggtagaacgacactgggcggagccaccccagggcctgctcccagggagtgctcgaggcgcatcaggcccgttttttaccagtttatatcacggtcttcatttttaaaagtaacgctaactttgtacggacgatgtctcatggattaaataatattctttatggca

SEQ ID NO: 8 - DNA, 3613 bp, synthetic construct
  source 1..3613: mol_type = other DNA
actctgctctcagccgcttcccgggacgtggggcctccgagaggttggcctgcttgggcggctgccctgcctgacccacagccatctgtcagtccatctgtccgccttgaccccaggagacccagagatcgaggaaaggatcggagcatggtgtagacaccctcagccagcccagggagcccggccccgcacatctgagggaaggaagtggctggccaggtcctgcggcccctctggatgcccgtgctggcctctcctctagcatcgtgcccaccatggcctcagctgacaagaatggcagcaacctcccatctgtgtctggtagccgcctgcagagccggaagccacccaacctctccatcaccatcccgccaccagagagccaggcccccggcgagcaggatagcatgcttcctgagaggcgcaagaacccagcctacctgaagagtgtcagcctacaggagccccggggacgatggcaggagggcgcagagaagcgccccggcttccgccgccaggcctccctgtcccagagcatccgcaagagcacagcccagtggtttggggtcagcggcgactgggagggcaagcgacaaaactggcatcgtcgcagcctgcaccactgcagcgtgcactatggccgcctcaaggcctcgtgccagagagaactggagctgcccagccaggaggtgccatccttccagggcactgagtctccaaaaccgtgcaagatgcccaagattgtggatccactggctcggggtagggccttccgccatccagatgaggtggaccggcctcacgctgcccacccacctctgactccaggggtcctgtctctcacatccttcaccagtgtccgctctggctactcccatctgccccgccgcaagaggatatctgttgcccatatgagctttcaggcagccgccgccctcctcaaggggcgttccgtgctagatgcgactgggcagcggtgccggcatgtcaaacgcagcttcgcttaccccagcttcctggaggaggatgctgtcgatggagctgacaccttcgactcctccttttttagtaaggaagaaatgagctccatgcctgacgatgtctttgagtcccccccactctctgccagctacttccgaggtgtcccacactctgcctccccggtctccccggatggagtgcacatcccgctaaaagaatacagcggtggccgagccctgggtcccgggacccagcgtggcaaacgcattgcctccaaagtaaagcactttgcatttgaccggaagaagaggcactacggcctgggcgtcgtgggtaactggctcaaccgaagctatcgacgcagcatcagcagcaccgtgcagcggcagctggagagcttcgatagccaccggccctacttcacctactggctgacgttcgttcacatcatcatcaccttgctggtgatctgcacctatggcatcgcacctgtgggctttgcccagcacgttaccacccagctggtgctgaagaacagaggcgtgtatgagagcgtgaagtacatccagcaggagaacttctggattggccccagctcgattgacctcattcacctgggagcaaagttctcgccctgcatccggaaggaccagcaaattgagcagctggtacggagggagcgcgacattgagcgcacctctggctgctgtgtccagaatgaccgctcgggctgcatccagaccctgaagaaggactgctcggagactttagccacgttcgtaaagtggcagaatgatactgggccctcagacaagtctgacctgagccagaagcagccatcggcggttgtgtgccaccaagaccccaggacctgtgaagagcctgcctccagtggggcccacatctggcctgatgacattaccaagtggccgatctgcacagagcaggctcagagcaaccacacgggcttgttgcacatagactgtaagatcaaaggccgcccctgctgcatcggcaccaagggcagctgcgagatcaccactcgggagtactgtgagttcatgcatggctatttccatgaagacgcgacgctgtgttcccaggtgcactgtttagacaaggtgtgtgggctcctgcctttcctcaaccctgaggtccctgaccagttctaccggatctggctgtctttattcctgcatgctggcatagtgcactgccttgtgtctgtggtcttccaaatgaccatcctgagggacctagagaagctggccggctggcaccgcatctccatcatcttcatccttagtggcattacaggcaacctggccagcgccatcttcctcccctaccgggcagaggtgggcccagccgggtcgcagttcggcctcctcgcctgcctcttcgtggagctgttccagagctggcagctgttggagcggccgtggaaggccttcttcaacctgtcggccattgtgcttttcctcttcatctgtggcctcctgccctggatagacaacatcgcccacatcttcgggttcctcagcggcatgcttctggccttcgccttcctgccttacattaccttcggcaccagcgacaagtaccgcaagcgagccctcatcctcgtgtcgctgctggtctttgctgggctctttgcttccctggtgctgtggctgtacatctaccccatcaactggccctggatcgagtacctcacctgctttcccttcaccagccgcttctgtgagaagtacgagctagaccaggtgctacactaactgcagagattgtgtgtctgccctgggccgtgtgtctatgaaccggtggggccctgagcccagcagctcggtccacactcaaggctgactccagatgagacgggcggtaaaggcaggctccccagggagatgactcctcctttctcaggtctgaatgttcctgacccaagtctgggggacatccaggacacttgcttctctgaggctcaggtcccaggccctgcctgcctctctggctctataaagatgataacttttcttgggcctctggcctctgcgggtgctgtctccccactgacactgtgactgtgacctcgccagacacacagctgctctctcagttgtccccagggttgaggtccttaccatgctgctatgaccccgttttcctgtttctcctctcctccctgtccttcctgtgtttgtccgtggactcgtgagcctgctcctgaggctcctggacatagagcattgtggggaaggctctggctgttgtctatgggggatgacaagcaaggagagatggctatacagggatgctaggggcttttgttaagcaaagaagccagctctcctaagcccatcagctgccctagctccagcatgtgtctggctgcacaggttggctctgatcccaggatgcccctgccacctgccctcactcctgcgtggccgtgggccgagccgccttgaggactagctcccagaggaggcctgaggcccagactggtgggttttttggttttgttttgtttttttttttccaatttatattatggtcctaattttgtaaagtaacgctaactttgtacggatgatgtctcaagtttattaaatgacattctttattaaaa

SEQ ID NO: 9 - DNA, 493 bp, synthetic construct
  source 1..493: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 10 - DNA, 283 bp, synthetic construct
  source 1..283: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 11 - DNA, 24 bp, synthetic construct
  source 1..24: mol_type = other DNA
cacccaactcgctgttgcggaatc

SEQ ID NO: 12 - DNA, 24 bp, synthetic construct
  source 1..24: mol_type = other DNA
aaacgattccgcaacagcgagttg

SEQ ID NO: 13 - DNA, 21 bp, synthetic construct
  source 1..21: mol_type = other DNA
ttcccgtcaaaggaagtttgg

SEQ ID NO: 14 - DNA, 21 bp, synthetic construct
  source 1..21: mol_type = other DNA
tatgatggactcatgccttgc

(19) United States
(12) Patent Application Publication
(10) Pub. No.: US 2024/0293579 A1
(43) Pub. Date: Sep. 5, 2024

(54) Composition Comprising Trim31 and Rhbdf2 for Treatment of NAFLD/NASH

(71) Applicant: CHONGQING MEIPULAN TECHNOLOGY CO., LTD, Chongqing (CN)

(72) Inventors: Jun TAN, Chongqing (CN); Minxuan XU, Chongqing (CN); Chenxu GE, Chongqing (CN); Yan SUN, Chongqing (CN); Xianling DAI, Chongqing (CN); Qin KUANG, Chongqing (CN); Junjie ZHAO, Chongqing (CN)

(73) Assignee: CHONGQING MEIPULAN TECHNOLOGY CO., LTD, Chongqing (CN)

(21) Appl. No.: 18/663,136

(22) Filed: May 14, 2024

Related U.S. Application Data
(63) Continuation-in-part of application No. PCT/CN2022/128995, filed on Nov. 9, 2022.

(30) Foreign Application Priority Data
Nov. 17, 2021 (CN) 202111361919.X

(51) Int. Cl. A61K 48/00; A61K 38/17; A61K 45/06; A61P 1/16; C12N 15/86

(52) U.S. Cl. CPC A61K 48/005; A61K 38/1709; A61K 45/06; A61P 1/16; C12N 15/86; C12N 2740/15043; C12N 2750/14143

(57) ABSTRACT

Methods and compositions for treating or preventing nonalcoholic fatty liver disease (NAFLD) or non-alcoholic steatohepatitis (NASH) are provided, including tripartite motif-containing protein 31 (TRIM31) and rhomboid 5 homolog 2 protein (RHBDF2), in particular a method for promoting the degradation of RHBDF2 by E3 ligase TRIM31. The invention also provides methods for treating NAFLD/NASH by reagents promoting TRIM31 expression or inhibit RHBDF2 expression, comprising recombinant lentiviral vector, adeno-associated virual vector, compounds, extracts of traditional Chinese medicine, etc.

### CROSS REFERENCE TO THE RELATED APPLICATIONS

This application is a continuation-in-part application of the national phase entry of International Application No. PCT/CN2022/128995, filed on Nov. 9, 2022, which is based upon and claims priority to Chinese Patent Application No. 202111361919.X, filed on Nov. 17, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBYBCQ001-PKG_Sequence_Listing, created on Apr. 30, 2024, and is 27,809 bytes in size.

### TECHNICAL FIELD

The present invention is in the field of biological medicine. In particular a therapeutic target and suitable compounds for treatment of non-alcoholic steatohepatitis (NASH) are provided.

### BACKGROUND

The latest epidemiological studies have found that obesity and its metabolic complications have become one of the most serious public health crises. With the increase in high-risk obesity populations, continuous and prolonged high-energy diet intake increases the risk of systemic metabolic syndrome, including obesity, hyperlipidemia, and severe cardiovascular and cerebrovascular diseases. In fact, the prevalence of obesity-related comorbidities such as non-alcoholic fatty liver disease (NAFLD), has increased in parallel, and NAFLD is already the most common chronic liver disease. The severity of NAFLD ranges from simple steatosis to hepatocyte injury with malignant transformation and necrotizing inflammatory alterations characterized as nonalcoholic steatohepatitis (NASH), which renders patients more susceptible to liver fibrosis and hepatocellular carcinoma. Thus far, unfortunately, there are no approved effective therapeutic strategies for NASH in the world, and methods to alleviate the related complications induced by this disease do not fully meet expectations. The development of effective drugs and therapeutic options for NAFLD/NASH mainly depends on targeting pivotal signaling regulators or regulatory processes closely related to pathogenic mechanisms. Given that the pathological process of NAFLD and NASH is a tangled progression associated with metabolic syndrome and systemic inflammatory response syndrome (SIRS), the common targets should be identified to manipulate the pathogenic signaling pathways.

Rhomboid 5 homolog 2 (Rhbdf2), also known as iRhom2, is an inactive member of the rhomboid intramembrane proteinase family that has been determined to be a pivotal pathogenic regulator of inflammation-related diseases, e.g., obesity, arthritis, nephritis, atherosclerosis, and fibrosis. Rhbdf2 recruits MAP3K7 to significantly increase its phosphorylation levels and activate downstream inflammatory signaling. The activation of Rhbdf2-MAP3K7 signaling contributes to occurrence of NAFLD, which predisposes pathological phenotypes to hepatic fibrosis.

E3 ubiquitin ligase-tripartite motif containing protein 31 (Trim31) has been identified as a “Janus-faced” regulator of innate immune responses by facilitating the targeted substrate degradation or signal transduction via ubiquitin modification. Moreover, the role of Trim31, especially whether TRIM31 regulates RHBDF2 ubiquitination and RHBDF2-mediated NAFLD/NASH pathogenesis remains unknown, remains unknown. Therefore, the search for key signal modulators in the pathogenesis of NASH has become an urgent problem in drug development for NASH.

### SUMMARY

The invention provides a composition comprising E3 ubiquitin ligase-tripartite motif containing protein 31 (Trim31) and Rhomboid 5 homolog 2 (Rhbdf2) derived from hepatocytes. The TRIM31 preferably comprises an amino acid sequence as shown in SEQ ID NO: 1 or SEQ ID NO: 2; The RHBDF2 preferably comprises an amino acid sequence as shown in SEQ ID NO: 3 or SEQ ID NO: 4. Thereinto, the nucleic acid sequences encoding TRIM31 include sequences such as SEQ ID NO: 5 or SEQ ID NO: 6; nucleic acid sequences encoding RHBDF2 include sequences such as SEQ ID NO: 7 or SEQ ID NO: 8.

In another embodiment, the nucleic acid sequence encoding TRIM31 is a humanized sequence shown in SEQ ID NO: 5; the nucleic acid sequence encoding RHBDF2 is a humanized sequence shown in SEQ ID NO: 7.

Further, there is an inverse relationship in quantity between the TRIM31 and the RHBDF2. In other words, if the secretion of TRIM31 is increased, the secretion of RHBDF2 is inhibited, that is, the secretion of RHBDF2 is decreased.

The invention provides an application of a composition comprising TRIM31 and RHBDF2 in the preparation of a drug for the treatment of nonalcoholic fatty liver disease (NAFLD).

The non-alcoholic fatty liver disease (NAFLD) includes but is not limited to disorders of liver lipid metabolism, simple fatty liver, non-alcoholic steatohepatitis (NASH), cirrhosis, etc., preferably non-alcoholic steatohepatitis (NASH).

The invention also provides a reagent that promotes the expression of TRIM31 or inhibits the expression of RHBDF2 used in the preparation of drugs for the treatment of NASH.

Further, the reagent comprises a recombinant lentiviral vector packaging Trim31 with a nucleic acid sequence such as SEQ ID NO: 5 or SEQ ID NO: 6 that the full-length Trim31 cDNA sequences. The vectors include but are not limited to pLentiCMV, pCMV, pLV, pcdna3.1, preferably pLentiCMV.

Further, in the another embodiment, the reagent comprises a recombinant adeno-associated virual vector packaging Trim31 with a nucleic acid sequence such as SEQ ID NO: 5 or SEQ ID NO: 6 that the full-length Trim31 sequences. The vectors include but are not limited to AAV1, AAV2, AAV3, AAV4, AAV5, AAV6, AAV7, AAV8, preferably AAV-TBG.

Further, the reagent comprises one or more combinations of the following substances:
(1) Alkaloid compounds or their compositions including magnoflorine, peganine, serpentine, leonurine, tetrandrine, orixine, peimine, colchicine, piperine, rhynchophylline, camptothecin, tetrahydropalmatine, jateorhizine, chelidonine, lycorine, acronycine, sophocarpidine, oxymatrine.
(2) Flavonoids or their compositions including quercetin, dihydroquercetin, luteolin, morin, Mulberrin, apigenin, diosmetin, chrysin, tricin, Puerarin, anthocyanin, proanthocyanidins, daidzein, baicalein, scutellarein, icariin, wogonin, cyanidin, silymarin, silibinin, genistein, galangin, kaempferol, fisetin, isorhamnetin, myricetin, hesperetin, naringenin, liquiritigenin, catechin, epicatechin, epigallocatechin, delphinidin, pelargonidin, breviscapine, afzelechin, rutin, luteolin-7-O-β-D-glucoside, baicalin, apiin, juglanin.
(3) Polyphenolic compounds or their compositions, including chlorogenic acid, cynarin, cynarascoloside C, cynaropicrin, tea polyphenols, resveratrol, punicosides, curcumin, ferulic acid
(4) Amine compounds or their compositions, including penicillamine, pomadomide, lenalidomide, thalidomide
(5) Extracts of traditional Chinese medicine, including Astragalus membranaceus, Codonopsis pilosula, Salvia miltiorrhiza, Angelica sinensis, Chinese yam, pseudo-ginseng, Schisandra chinensis, Polygonatum sibiricum, Ganoderma lucidum, Trametes versicolor, artichoke, Thistle, milk thistle, Pueraria lobate, Coptis chinensis, Phellodendron amurense, turmeric, Honeysuckle, Raisin Tree Seed, pericarpium citri reticulatae, mulberry leaf, gingko leaf, lily, bear bile, pig bile, Yinchen, Fructus aurantii, Licorice, Polygonum cuspidatum, Sophora japonica, the root bark of the peony tree, Sedum sarmentosum, gardenia, Chinese Wolfberry, White Peony, Ligusticum wallichii, peach kernel, rheum officinale, chrysanthemum, serrate rabdosia herb, Hedyotis diffusa, the root of fangji, white grass root, Sculellaria barbata, radix bupleuri, Poria cocos.
(6) other compounds or their compositions, including Glycyrrhizinic acid, Glycyrrhizic acid monoammonium salt, adenosine, ursodeoxycholic acid, tauroursodeoxycholic acid, chenodeoxycholic acid, taurochenodeoxycholic acid, dicyclol, bifendate, sorafenib, regorafenib, levatinib, betacyanin, metformin, carminic acid.

Further, the reagent includes a number of pharmaceutical excipients

The invention also provides a method for promoting the degradation of RHBDF2 in the preparation of drugs for the treatment of NASH, characterized in that the TRIM31 directly interactions with RHBDF2 to promote the degradation of RHBDF2 through K48-linked polyubiquitination.

Further, the method includes a reagent that brings together the E3 ligase and RHBDF2 to promote RHBDF2 degradation via ubiquitination. E3 ligases include but are not limited to TRIM31, CRBN, VHL, TRIM26, TRIM38, etc, preferably TRIM31.

Specifically, the invention reveals that Trim31 is a crucial eliminator of genetically and high-energy diet-triggered insulin resistance, liver steatosis, inflammation, and hepatic fibrosis by promoting degradation of Rhbdf2 by K48-linked polyubiquitination, which results in suppression of Rhbdf2-MAP3K7 signaling and downstream events.

The invention also reveals that K48-linked Rhbdf2 ubiquitination could be catalyzed in vitro, and Trim31 deletion greatly blocked this endogenous ubiquitination of Rhbdf2. More detailed in vitro studies of the molecular biological functions of Trim31 have shown that it directly binds to Rhbdf2 via the CC domain to target Rhbdf2 activity; the RING-finger of Trim31 ubiquitinates Rhbdf2, mediating proteasomal Rhbdf2 degradation, via the K48 linkage. Therefore, Trim31-Rhbdf2 binding and the subsequent Rhbdf2 ubiquitination are required for and conduce to the mitigation of liver steatosis, insulin resistance, and liver inflammation triggered by hepatocyte Rhbdf2-MAP3K7 signaling.

Our previous finding also indicated that the absence of Rhbdf2 in non-immune cells did not affect the expression and secretion of inflammatory factors such as TNF-α, IL-6 and IL-1β. Of note, although the release of TNF-α in non-immune cells, e.g., hepatocytes, does not completely depend on the trafficking of Rhbdf2, increased ubiquitination of Rhbdf2 is able to recruit MAP3K7 and elevate its phosphorylation levels, and then promote downstream cascades activation under stimulation conditions, significantly activate NF-κB inflammatory signals and accelerate the secretion of inflammatory factors. In the present invention, we have found that in PA-induced hepatocytes, Trim31 significantly promotes the degradation of Rhbdf2 by K48 ubiquitin linkage, followed by a strong decrease in the expression of TNF-α and other inflammatory factors.

The invention determined that liver Trim31 expression correlated with NAFLD/NASH and metabolic disorder e.g., insulin resistant and glycometabolic disorder both in humans and in murine. Furthermore, the hepatic Trim31 expression in mice is responsive to dietary interventions that significantly moderated insulin resistance, hepatic steatosis, and inflammation phenotype via mechanically, regulating Rhbdf2 proteasome degradation. Finally, these obtained findings encourage Trim31 as a feasible therapeutical target for NAFLD/NASH and associated-metabolic disorders, and the combination of TRIM31 and RHBDF2 can be used to prepare drugs for the treatment of NASH.

The invention has the beneficial effects as follows:

In the invention, the composition of TRIM31 and RHBDF2 plays a key role in liver metabolic disorders, hepatocellular degeneration, insulin resistance, inflammation, etc.

In the invention, the reagent for promoting TRIM31 expression can effectively improve NAFLD/NASH or metabolic disorders.

The invention provides a method for promoting RHBDF2 degradation, which can effectively relieve insulin resistance, liver steatosis or inflammation and liver fibrosis.

### BRIEF DESCRIPTION OF THE DRAWINGS

FIG. 1 Representative immunoblotting bands of Trim31 expression in the liver samples isolated from C57BL/6N mice that were treated with a NCD or HFD for uninterrupted 16 weeks, or from ob/ob or lean mice.

FIG. 2 Representative immunoblotting bands of Rhbdf2 expression in the liver samples isolated from C57BL/6N mice that were treated with a NCD or HFD for uninterrupted 16 weeks, or from ob/ob or lean mice

FIG. 3 Representative immunoblotting bands of Trim31 expression in the liver samples isolated from C57BL/6N mice that were treated with a HFD over time.

FIGS. 4A-4B Representative immunoblotting bands and relative expression levels of TRIM31 in the liver samples of donors with non-steatosis, simple steatosis or NASH phenotype.

FIGS. 5A-5B Representative immunoblotting bands and relative expression levels of RHBDF2 in the liver samples of donors with non-steatosis, simple steatosis or NASH phenotype.

FIGS. 6A-6B Representative immunoblotting bands of Trim31 or Rhbdf2 expression in cultured primary hepatocytes that were incubated with 400 μM palmitate (PA) or 100 ng/ml TNF-α, respectively.

FIG. 7 Representative immunoblotting bands of Rhbdf2 expression in cultured primary hepatocytes that were incubated with 400 μM palmitate (PA). The BSA was treated as controls

FIGS. 8A-8C Representative western blotting for the Trim31 expression in different organs (upper), and expression in the liver samples from Trim31-Flox and hepatocyte-specific Trim31 deletion (THKO) mice (lower).

FIGS. 9A-9C Representative western blotting for the Trim31 expression in different organs (upper) and in the liver samples from non-transgenic Trim31 (NTG) and THTG mice (lower).

FIGS. 10A-10D Body weight records, fasting blood glucose levels, of non-transgenic Trim31 (NTG) and hepatocyte-specific Trim31 transgenic (THTG) mice during 16 weeks of NCD or HFD administration (upper). Fasting insulin levels and corresponding HOMA-IR index of NTG and THTG mice at the last week of NCD or HFD feeding (lower).

FIG. 11 Representative images of Periodic Acid-Schiff stain (PAS)-stained pathological section for glycogen storage changes in the liver samples from the Flox-and THKO-mice (TRIM31 knockout).

FIG. 12 Representative images of Periodic Acid-Schiff stain (PAS)-stained pathological section for glycogen storage changes in the liver samples from the NTG and THTG mice (TRIM31 overexpression)

FIG. 13 Representative pictures for liver appearance and transmission electron microscope (TEM)-indicated histological changes of the liver in Flox-and THKO-mice (TRIM31 knockout) after NCD or HFD feeding for 16 weeks.

FIGS. 14A-14B Liver lipid contents including triglyceride (TG), total cholesterol (TC) and non-esterified fatty acids (NEFA) (FIG. 14A), and serum alanine transaminase (ALT), aspartate aminotransferase (AST) and alkaline phosphatase (AKP) levels (FIG. 14B) of the Flox-and THKO-mice after HFD treatment for 16 weeks.

FIG. 15 Representative pictures of H&E-stained (upper) and Oil-red O-stained (lower) pathological section of the liver from the 16-weeks HFD-fed Flox-and THKO-mice (TRIM31 knockout).

FIG. 16 qPCR analysis of the relative mRNA expression of genes associated with fatty acid uptake, synthesis, and β-oxidation in Flox-and THKO-mice after 16-weeks HFD feeding.

FIG. 17 Representative pictures for liver appearance and TEM-indicated histological changes of the liver in NTG-and THTG-mice (TRIM31 overexpression) after 16-weeks NCD or HFD feeding

FIGS. 18A-18B Liver lipid contents including TG, TC and NEFA (FIG. 18A), and serum ALT, AST and AKP levels (FIG. 18B) of the NTG- and THTG-mice after HFD treatment for 16 weeks.

FIG. 19 Representative pictures of H&E-stained (upper) and Oil-red O-stained (lower) pathological section of the liver from the 16-weeks HFD-fed NTG- and THTG-mice (TRIM31 overexpression).

FIG. 20 qPCR analysis of the relative mRNA expression of genes associated with fatty acid uptake, synthesis, and β-oxidation in NTG- and THTG-mice (TRIM31 overexpression) after 16-weeks HFD feeding.

FIG. 21 Representative immunoblotting bands for expression alterations of total amounts or phosphorylated forms of critical indicators associating with inflammation signaling including IKKβ, p-IKKβ, IκBα, p-IκBα, NF-κB and p-NF-κB in the liver samples from THKO-, THTG-, NTG-, Flox mice after 16-weeks HFD or NCD feeding.

FIGS. 22A-22F qPCR analysis of the relative mRNA expression of genes associated with inflammation signaling in the liver samples from THKO-, THTG-, NTG-, Flox mice after 16-weeks HFD or NCD feeding.

FIGS. 23A-23B Representative pro-inflammatory cytokines and chemokines levels including TNF-α, IL-1β, IL-6, CCL-2, and IL-10 in serum from THKO-, THTG-, NTG-, Flox mice after 16-weeks HFD or NCD feeding.

FIG. 24 Representative pictures for liver appearance and Oil red O staining, H&E staining, and PAS staining-indicated liver histopathologic changes of the HFD-fed recipient (THKO)(LV−) and (THKO)(LV+) mice after ex vivo experiment.

FIG. 25 Liver function markers ALT and AST levels were detected in the HFD-fed recipient (THKO)(LV−) and (THKO)(LV+) mice after ex vivo experiment

FIGS. 26A-26C Records for the glucose tolerance test (GTT), insulin tolerance test (ITT), and the corresponding fasting insulin levels and HOMA-IR index in the HFD-fed recipient (THKO)(LV−) and (THKO)(LV+) mice after transplantation.

FIG. 27 Liver lipid contents including TG, TC and NEFA of the HFD-fed recipient (THKO)(LV−) and (THKO)(LV+) mice after transplantation.

FIG. 28 qPCR analysis of the relative mRNA expression of genes associated with fatty acid uptake, synthesis, and β-oxidation in the HFD-fed recipient (THKO)(LV−) and (THKO)(LV+) mice after transplantation.

FIGS. 29A-29B Representative immunoblotting bands for expression alterations of total amounts and phosphorylated forms of critical indicators involving in insulin signaling, including IRS1, p-IRS1(Ser307), p-IRS1(Tyr608), AKT, and p-AKT in the liver of HFD-fed recipient (THKO)(LV−) and (THKO)(LV+) mice.

FIGS. 30A-30B Representative immunoblotting bands for expression alterations of total amounts and phosphorylated forms of critical indicators involving in inflammation signaling, including Rhbdf2-MAP3K7 axis and p-NF-κB pathway in the liver of HFD-fed recipient (THKO)(LV−) and (THKO)(LV+) mice.

FIG. 31 Preconditioned liver-specific Trim31 deletion (THKO) mice with an 8-weeks HFD treatment as donor were transduced (THKO)(LV−). The hepatocytes isolated from (THKO)(LV−) group mice were transduced with lentivirus-loaded human full-length TRIM31 sequence. The corresponding blank vector were transduced as controls. Then, the additional HFD-fed THKO mice as recipient were injected with transduced hepatocytes via portal vein. The HFD-fed transplanted (THKO)(LV+) mice were harvested for the further experimental detection.

FIG. 32 Representative pictures for Oil red O staining and H&E staining-indicated liver histopathologic changes of the HFD-fed recipient (THKO)(LV−) and (THKO)(LV+) mice after ex vivo experiment.

FIG. 33 Liver function markers ALT and AST levels were detected in the HFD-fed recipient (THKO)(LV−) and (THKO)(LV+) mice after ex vivo experiment.

FIGS. 34A-34C Records for the glucose tolerance test (GTT) and insulin tolerance test (ITT), and the corresponding fasting insulin levels and HOMA-IR index in the HFD-fed recipient (THKO)(LV−) and (THKO)(LV+) mice after transplantation.

FIG. 35 Liver lipid contents including TG, TC and NEFA in the HFD-fed recipient (THKO)(LV−) and (THKO)(LV+) mice after transplantation.

FIG. 36 qPCR analysis of the relative mRNA expression of genes associated with fatty acid uptake, synthesis, and β-oxidation in the HFD-fed recipient (THKO)(LV−) and (THKO)(LV+) mice after transplantation.

FIG. 37 Representative immunoblotting bands for expression alterations of total amounts and phosphorylated forms of critical indicators involving in insulin signaling, including IRS1, p-IRS1(Ser307), p-IRS1(Tyr608), AKT, and p-AKT, and Rhbdf2-MAP3K7 axis and p-NF-κB pathway in the liver of HFD-fed recipient (THKO)(LV−) and (THKO)(LV+) mice.

FIG. 38 Pro-inflammatory cytokines levels (TNF-α, IL-6 and IL-1β) in serum in the liver of HFD-fed recipient (THKO)(LV−) and (THKO)(LV+) mice.

FIG. 39 Representative immunofluorescence images of Rhbdf2 and p-MAP3K7 co-expression in mice liver sections isolated from Flox-, THKO-NTG-and THTG-mice that were treated with a HFD for 16 weeks.

FIG. 40 Representative immunoblotting bands for expression alterations of total amounts or phosphorylated forms of critical indicators associating with the Rhbdf2-MAP3K7 axis and its downstream events cascades including ADAM17, TNFR1/2, MKK7, p-MKK7, c-Jun and p-c-Jun (Ser63) in the liver of a 16-weeks HFD-fed Flox-, THKO-NTG-and THTG-mice.

FIG. 41 Representative immunoblotting bands for expression changes of total amounts or phosphorylated forms of critical indicators associating with the insulin signaling, including IRS1, p-IRS1(Ser307), AKT, p-AKT, GSK3β, p-GSK3β, FOXO1 and p-FOXO1, in the adenovirus-packed full-length Rhbdf2 sequences (AdRhbdf2) or shRNA targeting Rhbdf2 (AdshRhbdf2)-transfected THKO or THTG primary hepatocytes that were treated with 400 μM PA for 10 hours.

FIG. 42 The primary hepatocytes isolated from the THKO or THTG mice were transfected with AdRhbdf2 or AdshRhbdf2. The upper western blotting bands shows the expression efficiency of Rhbdf2. The lower western blotting bands shows the inhibitory efficiency of Rhbdf2.

FIG. 43 Representative western blotting for co-immunoprecipitation (CO-IP) detection in L02 cells transfected with HA-or Flag-tagged RHBDF2 or TRIM31 vectors. The anti-Flag or anti-HA antibodies were used as probes.

FIG. 44 The binding domains of RHBDF2 and TRIM31 were detected using full-length and truncated Rhbdf2 or Trim31 expression vectors based on IP assays. Anti-Flag or anti-HA antibodies were used to confirm the binding sites of RHBDF2 and TRIM31, respectively.

FIG. 45 The binding domains of mice Rhbdf2 and Trim31 were detected using full-length and truncated Rhbdf2 or Trim31 expression vectors based on IP assays. Anti-Flag or anti-HA antibodies were used to confirm the binding sites of Rhbdf2 and Trim31, respectively.

FIG. 46 The ubiquitination levels of Rhbdf2 in the liver samples of Trim31-Flox or THKO mice in the presence of HFD feeding for 16 weeks.

FIG. 47 Representative western blotting assays of lysates from L02 cells transfected with Myc-tagged ubiquitin (Myc-Ub), RHBDF2-HA, full-length TRIM31-Flag and TRIM31 with RING domain mutant (TRIM31-Flag RING^{Δ}), followed by IP with anti-HA, probed with K48-Ub or anti-Myc.

FIGS. 48A-48B Ubiquitination levels of RHBDF2 after TRIM31-Flag overexpression and in response to PA administration in L02 cells co-transfected with RHBDF2-HA and the indicated Myc-tagged ubiquitin constructs (K48O, K63O, K33O, K6O, K29O, K27O) (FIG. 48A). The K48O means ubiquitin in which all lysines except K48 were mutated. The empty vector was used as control. Representative western blotting indicating the ubiquitination levels of RHBDF2 in L02 cells transfected with the K48O vector in different combinations and the indicated downstream events cascades protein expression levels in WCL (FIG. 48B).

FIG. 49 Representative images of the intracellular TG levels in THKO-L02 cells transfected with adenovirus-packed full-length TRIM31 sequences (AdTRIM31) or different truncated TRIM31 sequences, which were then incubated with 500 μM PA for 4 hours. The adenovirus-containing GFP vector (AdGFP) was used as controls.

FIG. 50 Representative immunoblotting bands of Trim31, Rhbdf2 and corresponding downstream events indicators levels of total and phosphorylated ADAM17, MAP3K7, p-MAP3K7, NF-κB, p-NF-κB, IκBα, p-IκBα, JNK1/2 and p-JNK1/2 in liver samples isolated from the indicated mice fed a HFD for 16 weeks.

FIG. 51 Records for body weight, liver weight, ratio of liver weight/body weight (%) in a 16-weeks HFD-fed indicated mice.

FIG. 52 Records for fasting blood glucose levels, fasting insulin levels and HOMA-IR index in a 16-weeks HFD-fed indicated mice.

FIG. 53 Representative pictures for Oil red O staining and H&E staining-indicated liver histopathologic changes of a 16-weeks HFD-fed indicated mice

FIG. 54 Liver lipid contents including TG, TC and NEFA of the indicated mice after a 16-weeks HFD ingestion.

FIG. 55 qPCR analysis of the relative mRNA expression of genes in the livers associated with inflammation signaling in 16-weeks HFD-fed mice.

FIG. 56 Representative images of Oil red O staining of primary hepatocytes that were transfected with AdshRhbdf2 or AdshTrim31 or co-treated with AdshRhbdf2/AdshTrim31 or PA for 10 hours

FIG. 57 Records for liver weight, ratio of liver weight/body weight in THTG and NTG mice after a 16-weeks HFHF administration.

FIG. 58 Records for time-course changes of body weight in THTG and NTG mice after a 16-weeks HFHF administration.

FIG. 59 Liver lipid contents including TG, TC and NEFA of the indicated mice after a 16-weeks HFHF ingestion.

FIG. 60 Representative pictures for Oil red O staining and H&E staining-indicated liver histopathologic changes of a 16-weeks HFHF-fed THTG and NTG mice.

FIG. 61 qPCR analysis of the relative mRNA expression of genes in the livers associated with fatty acid metabolism after a 16-weeks HFHF administration.

FIG. 62 Immunohistochemistry staining of F4/80 analysis showing the histopathologic changes of the liver samples in indicated mice.

FIG. 63 qPCR analysis of the relative mRNA expression of genes in the livers associated with inflammation after a 16-weeks HFHF administration.

FIG. 64 Representative pictures for Sirius red staining and Masson staining showing the collagen deposition levels of the liver samples in indicated mice.

FIG. 65 PCR analysis of the relative mRNA expression of genes in the livers associated with collagen synthesis after a 16-weeks HFHF administration.

FIG. 66 Hepatic function indicator AST, ALT and AKP levels in the 16-weeks HFHF-fed THTG and NTG mice.

FIGS. 67A-67B Effect of betacyanin (BCN) on the relative expression of proteins in the livers associated with inflammation and fibrosis in THTG mice.

FIG. 68 Effect of fisetin (FIS) on the relative mRNA expression of genes in the livers associated with inflammation and fibrosis in high-fat-diet (HFD) mice.

FIG. 69 Effect of carminic acid on the relative mRNA expression of genes in the livers associated with inflammation and fibrosis in mice fed with fructose (Fru). CAL indicates low concentration of carmine acid, and CAH indicates high concentration of carmine acid

### DETAILED DESCRIPTION OF THE EMBODIMENTS

In the invention, the embodiments given are intended to better illustrate the invention, but the content of the invention is not limited to the embodiments given. Therefore, the non-essential improvement and adjustment of the implementation scheme by the technical personnel familiar with the field according to the invention content is still within the scope of protection of the invention.

### Animals

Trim31^{flox/flox} mice based on C57BL/6N background were generated using CRISPR/Cas9-mediated genome engineering system. Exons 4 and 5 of Trim31 were then selected as conditional knockout region (CKO). In brief, the chosen exons of Trim31 were flanked by loxP sites, and therefore two single guide RNAs (gRNA1 and gRNA2) targeting Trim31 introns were designed. The targeting vector containing Trim31 exon 4 and 5 flanked by two loxP sites and the two homology arms were used as the template. The targeting vector, guide RNAI and guide RNA2 and Cas9 mRNAs were co-injected into fertilized eggs for CKO mouse production. The obtained mice, which had exon 4 and 5 flanked by two loxP sites on one allele, were used to construct Trim31^{flox/flox} mice. Hepatocyte-specific Trim31 deletion (THKO) mice were created by mating Trim31^{flox/flox} mice with albumin-Cre (Alb-Cre) mice (Jackson Laboratory, Bar Harbor, Maine, USA). Trim31^{flox/flox} mice littermates were used in the study as controls for the obtained THKO mice.

The hepatocyte-specific Rhbdf2-knockout (RHKO) mice were also created using CRISPR/Cas9 system by specifically ablating the 4^{th} exon of Rhbdf2 in hepatocytes. Detailed protocols and information regarding the establishment and genotype determination of these mice have been described in our previous report (Xu Min-Xuan, Tan Jun, et al., Journal of Hazardous Materials, 2020, 400: 123158). In addition, the hepatocyte-specific Trim31 and Rhbdf2 double deletion (DHKO) mice were generated by crossing Trim31^{flox/flox} mice with RHKO mice.

Conditional Trim31 transgenic (TG) mice were established by micro-injecting CAG-loxP-CAT-loxP-Trim31 into fertilized eggs isolated from C57BL/6 mice. The obtained pups were then genotyped by PCR followed by sequencing analysis. The obtained mice were identified by PCR analysis of tail genomic DNA. The offspring of these TG mice were mated with the Alb-Cre mice to establish hepatocyte-specific Trim31 transgenic (THTG) mice. The corresponding littermates without Trim31 overexpression in hepatocytes were used as controls (NTG).

All the other normal wild-type (WT) C57BL/6N mice used in the current study were purchased from Beijing Vital River Laboratory Animal Technology Co., Ltd. (Beijing, China).

### Animals Experiment Design

All animal procedures and protocols were approved by the Animal Care and Use Committee of all participating Units. Prior to all experiments proper starts, the mice were subjected to adapt to the living environment for 7 days. The mice were housed in a constant temperature, humidity (controlled by GREE central air-conditioner, #GMV-Pd250W/NaB-N1, China) and pathogen-free-controlled environment (25° C.±2° C., 50%-60%) cage with a standard 12 h light/12 h dark cycle, plenty of water and food (pathogen-free) in their cages.

The 6-8-week-old WT male mice (total 15) were fed with high fat diet (HFD) fodder (20% kcal protein, 60 kcal % fat and 20% kcal carbohydrate, #D12492; Research Diets, New Brunswick, NJ, USA) for 16 weeks to induce fatty liver. The additional WT mice (total 15) were fed with a standard normal chow diet (20% kcal protein, 10 kcal % fat and 70% kcal carbohydrate, #D12450H; Research Diets, New Brunswick, NJ, USA) for 16 weeks to be served as control group (NCD). The age-matched (6-8-week-old) ob/ob mice (total 10) (#N000103, Nanjing Biomedical Research Institute of Nanjing University) were fed with NCD and then treated as another fatty liver model. In time-course experiments, a total of 10 WT mice for each time point were included. At the end of experimental period, the liver tissue samples were collected from mice to detect corresponding signaling events.

To investigate the protective function of Trim31 on HFD-induced insulin resistance, hepatic steatosis and inflammation, the ex vivo gene therapy interventions by lentivirus-loading full-length Trim31 sequences (LV-Trim31) or mutant Trim31 with RING domain deletion (LV-Trim31 RING^{Δ}) transduction and transplantion were performed in 8-weeks HFD-fed preconditioned THKO mice. The detailed protocols of the ex vivo therapy experiments were established in accordance with our previous reports (Xu, M. et al. Hepatology 2021, 73, 1346-1364).

To specifically over-expressed Trim31 in hepatocytes in vivo experiments, the adeno-associated virus serotype 8 (AAV8)-thyroxine-binding globulin (TBG) encoding full-length Trim31 sequences (AAV-Trim31) and AAV8 encoding mutant Trim31 with RING domain deletion (AAV-Trim31 RING^{Δ}) delivery system were established according to a standard molecular procedure. Briefly, the whole opening reading frame (ORF) encoding Trim31 without intervening stop codon was cloned into AAV8 vector to generate AAV-Trim31. Accordingly, 6-weeks HFD-fed preconditioned WT mice were injected with AAV-Trim31 or AAV-Trim31 RING^{Δ} by tail vein with 100 μl of virus containing 2×10¹¹ vg of vectors and then fed with HFD for additional 10 weeks. The empty vector (AAV-GFP) was injected into mice as the corresponding control.

To establish a fatty liver model, the male THKO, RHKO, DHKO, THTG mice and their corresponding littermates control mice at the age of 6-8 weeks were fed with HFD diet for 16 weeks to investigate the pathological changes. Also, the age-matched THKO, RHKO, DHKO, THTG mice and littermates were separately fed with NCD fodder for 16 weeks and treated as controls.

To further investigate the protective effects of Trim31 on liver steatosis, a nonalcoholic steatohepatitis (NASH) mice model was established in accordance with previous reports (Xu Min-Xuan,Tan Jun, et al., Journal of Hazardous Materials, 2020, 400: 123158). The THTG mice were then fed with HFHF diet (14% protein, 42% fat, 44% carbohydrates, 0.2% cholesterol and with a total of 42 g/L of carbohydreate mixed in drinking water at a ratio of 55% fructose and 45% sucrose by weight) for 16 weeks to produce phenotype of NASH. Also, the NTG mice used in this design as controls and were synchronously allowed to ad libitum access to this diet for 16 weeks.

### Cell Culture and Treatment

The L02 cell line (Human normal hepatocyte cell line) was purchased from Wuhan Shanen Biotechnology Co., Ltd., Wuhan, China. All resuscitated cell lines used in the laboratory of the applicant of the invention were passaged no more than 30 times. Cell lines involving in experiments need to be tested for mycoplasma contamination by PCR analysis. The L02 cells were maintained in Dulbecco's Modified Eagle Medium (DMEM) (#A4192101, Thermo Fisher Scientific) containing 10% fetal bovine serum (#16140071, Gibco™) and 1% penicillin-streptomycin (#15140-122; Gibco™) and were incubated in a 5% CO₂, 37° C. water-jacket type cell incubator (Thermo Fisher Scientific).

Primary hepatocytes used in the current experiments were isolated and collected from corresponding experimental mice by liver perfusion method. Briefly, under painless anesthesia condition, mice abdominal cavity was opened. Therefore, the livers were carefully perfused with 1×liver perfusion medium (#17701-038, Gibco™) and 1×liver digest medium (#17703-034, Gibco™) via the portal vein. Then, 100 μm steel mesh was used to grind and filter the digested liver tissue. The primary hepatocytes were collected by centrifuging the filter liquor at 800 rpm, 4° C. for 5 min, and further purified with 50% percoll solution (#17-0891-01, GE Healthcare Life Sciences). The obtained hepatocytes were cultured in DMEM medium containing 10% fetal bovine serum and 1% penicillin-streptomycin and cultured in a 5% CO₂, 37° C. cell incubator.

To construct a cell model of lipid deposition in vitro, corresponding concentration of palmitic acid (PA) (dissolved in 0.5% fatty acid-free BSA) was prepared and obtained. Then, the primary hepatocytes or L02 cells were treated with cell culture medium-containing PA for 10 h or 4 h. Fatty acid-free BSA (0.5%) alone was used as a vehicle control.

### Establishment of Knockout Cell Lines

The generation and protocol of Trim31-deficient cell lines used in this study were performed. In brief, cell lines with Trim31 deletion were produced by CRISPR/Cas9 gene editing system. The sgRNA targeting the human Trim31 genes were produced and packed into lentiCRISPR-V2 vectors to form the Cas9-sgRNA lentivirus. The oligo sequences used for generation of sgRNA expression vector are showed as: sgRNA-F: CACCCAACTCGCTGTTGCGGAATC (SEQ ID NO: 11); and sgRNA-R: AAACGATTCCGCAACAGCGAGTTG (SEQ ID NO: 12). The packaging vectors pSPAX2 and pMD2.G, together with sgRNA expression vector were then transfected separately into HEK293T cells using FuGENE® 6 Transfection Reagent for 42 hours. Next, the L02 cells were transduced with the obtained supernatant containing lentivirus to construct the gene knockout cell lines. The cell clones with target gene deletion were selected by immunoblotting.

### Plasmids Construction and Transfection

Human or mouse full-length Rhbdf2 and Trim31 expression plasmid were established by PCR-based amplification of cDNA, and then cloned into the 3×Flag-tagged pcDNA3.1 vector or 3×HA-tagged pcDNA3.1 vector (Invitrogen). Truncated Rhbdf2 and Trim31 fragments expression vector including TRIM31-Flag RING^{Δ}, TRIM31-Flag Box^{Δ}, TRIM31-Flag CC^{Δ}, RHBDF2-HA TMD^{Δ}, RHBDF2-HA IRHD^{Δ}, and RHBDF2-HA Tail^{Δ} as indicated in the figure legends, were obtained using standard PCR methods, and were then cloned into corresponding vectors. The Myc-ubiquitin WT expression vectors was constructed based on pcDNA3.1 vector. In addition, ubiquitin and corresponding derivatives including ubiquitin in which the only complete amino acid residue was ubiquitin-K48O, ubiquitin-K63O, ubiquitin-K33O, ubiquitin-K6O, ubiquitin-K29O, ubiquitin-K27O and control vector, were then packed into the Myc-tagged pcDNA3.1 plasmid (Thermo Fisher Scientific). Vectors were carefully transfected into L02 cells with Lipofectamine™ 3000 Transfection Reagent (Invitrogen™) according to the manufacturer's instructions.

Moreover, to further investigate the effects of Trim31 on lipid metabolism process in vitro experiments, here we have prepared an adenovirus-loaded Trim31 expression vector. Using a similar process to that of adeno-associated virus vector preparation, human full-length TRIM31 sequences and specific short hairpin RNA oligonucleotides sequences targeting human TRIM31 (shTRIM31) (shRNA sequences RNAi #1: TTCCCGTCAAAGGAAGTTTGG (SEQ ID NO: 13); RNAi #2: TATGATGGACTCATGCCTTGC (SEQ ID NO: 14)) were respectively packed into adenovirus (AdTRIM31; AdshTRIM31) by Easy Adenoviral Vector System Kit (#240009, Agilent Technologies). The AdshGFP was used as controls for knockdown or overexpression, respectively. The recombinant adenovirus was purified and titrated to 5×10¹⁰ plaque-forming units (PFU). The verification of the virus is based on DNA analysis of the virus, which is a plaque virus purified by restriction enzymes. After that, the hepatocytes were infected with adenovirus diluted in the culture medium, the number of infections was 50 times, and the infection was 24 hours.

### Adeno-Associated Virus and Lentivirus Construction and Production

The CAG promoter sequence is shown as SEQ ID NO: 9, and the CMV promoter sequence is shown as SEQ ID NO: 10.

In the embodiments of the invention, human TRIM31 expression gene is shown as SEQ ID NO: 5. Mouse TRIM31 expression gene is shown as SEQ ID NO: 6. Human RHBDF2 expression gene is shown as SEQ ID NO: 7. Mouse RHBDF2 expression gene is shown as SEQ ID NO: 8.

AAV8-TBG vector, a pre-packaged AAV in serotype 8 with overexpression of GFP, was used to produce recombinant AAV8-TBG-gene of interest-GFP expression vector. This vector contains transcriptional control elements from the thyroxine-binding globulin (TBG) promoter, cloning sites for the insertion of a complementary DNA and the polyA signal. Terminal repeats from AAV serotype 2 flank the expression cassette. The murine full-length Trim31 sequences or Trim31 with RING domain deletion sequences was then cloned into AAV8-TBG-GFP, respectively. This newly created vector AAV-TBG-Trim31-GFP or AAV-TBG-Trim31 RING^{Δ}-GFP was packaged into AAV8, purified by ViraBind™ AAV Purification Mega Kit (VPK-141/VPK-141-5, Cell Biolabs, VPK-141/VPK-141-5, San Diego, USA) and accordingly titered by QuickTiter™ AAV Quantitaiton Kit (Cell Biolabs, VPK-145). Viral particles were diluted to a total volume of 50 μl with saline immediately before injection.

To generate the lentiviral-Trim31 (LV-Trim31) or lentiviral-Trim31 with RING domain deletion (LV-Trim31 RING^{Δ}) vectors, the full-length Trim31 cDNA sequences were packaged into pLenti-CMV-GFP-Puro (Addgene) to upregulate Trim31 expression (pLenti-CMV-Trim31-GFP-Puro or pLenti-CMV-Trim31 RING^{Δ}-GFP-Puro) in vivo experiments. The commercial Lenti-Pac HIV Expression Packaging Kit (LT002, GeneCopoeia, MD, USA) and corresponding Lenti-Pac 293Ta Cell Line were used to produce LV particles. Next, according to the product instruction, the 293T cells culture supernatants containing virus particles were harvested. The newly created vector was concentrated and purified by ViraBindTM PLUS Lentivirus Concentration and Purification Kit (Cell Biolabs, VPK-095) and then titered by QuickTiter™ Lentivirus Quantitation Kit (Cell Biolabs, VPK-112). The functional LV titers in the 10⁶ TU/ml range were achieved, and after concentration yields of up to 10⁹ TU/ml were attained.

The immunoprecipitation assay was performed in the current study as follows. In brief, the L02 cells were transiently transfected with corresponding vectors in the indicated groups using Lipofectamine™ 3000 Transfection Reagent (Invitrogen™) according to the manufacturer's instructions and were cultured for an additional 36 h. Subsequently, the cells were harvested and homogenized into immunoprecipitation (IP)-specific lysis solution (#87787, Pierce™ IP Lysis Buffer, Thermo Scientific Pierce) at 4° C., followed by centrifugation at 13000 rpm for 20 min in a refrigerated centrifuge. The collected cell lysates were incubated with Protein A/G Magnetic Agarose Beads (#78609, Thermo Scientific Pierce) at room temperature with mixing for 2 hours, and then mixed with the indicated antibodies at 4° C. overnight. The immune compound was harvested after washing with immunoprecipitation buffer and subjected to western blotting assay by incubating with indicated primary antibodies and the corresponding secondary antibodies.

Direct protein interaction binding between Trim31 and Rhbdf2 was performed using the GST pull-down assays as follws. The Pierce™ GST Protein Interaction Pull-Down Kit (#21516, Thermo Fisher Scientific) was used to help with this part of the experiment. In brief, the Rosetta (DE3) E. coli cells were transformed with the plasmid pGEX-4T-1-GST-Trim31 or pGEX-4T-1-GST-Rhbdf2 and then induced expression by incubating with 0.5 mM isopropyl β-D-thiogalactopyranoside (IPTG) (#15502, Sigma-Aldrich). The extraction from lytic E. coli were mixed with GST beads at 4° C. for 1 h. The GST beads were then incubated with Flag-tagged Trim31 or Flag-tagged Rhbdf2, which were prepared by immunoprecipitation for the additional 4 h. Proteins that had interacted were eluted in elution buffer and were subjected to western blotting analysis using anti-Flag antibodies. The E. coli expressing only a GST-tag were used as the negative control.

Trim31, Rhbdf2 and Trim31 with RING domain deletion proteins were expressed with a TNT® Quick Coupled Transcription/Translation System (Promega) in accordance with manufacturer's instructions. Protein interaction binding assay were performed by mixing corresponding Flag-tagged Rhbdf2 and Trim31 together, followed by immunoprecipitation with Flag antibody and immunoblotting with Trim31 antibody. Ubiquitination levels was analyzed with a ubiquitination kit (Boston Biochem) following protocols of the manufacturer's instructions.

To perform immunoblotting analysis, cells or liver tissues were homogenized into RIPA Lysis and Extraction Buffer (#9806, CST) to yield a homogenate. Next, the final liquid supernatants were concentrated by centrifugation at 13500 rpm, 4°° C. for 30 min. Protein concentration was determined by Pierce™ Rapid Gold BCA Protein Assay Kit (Thermo Fisher Scientific) with bovine serum albumin as a standard. The total protein extraction samples were then subjected to western blotting analysis.

### Example 1 Trim31 Expression is Downregulated in Livers With Hepatic Steatosis

To determine whether Trim31 is involved in hepatic steatosis and metabolism, we first investigated its expression levels in liver tissues isolated from both dietary and obese mice model with steatohepatitis.

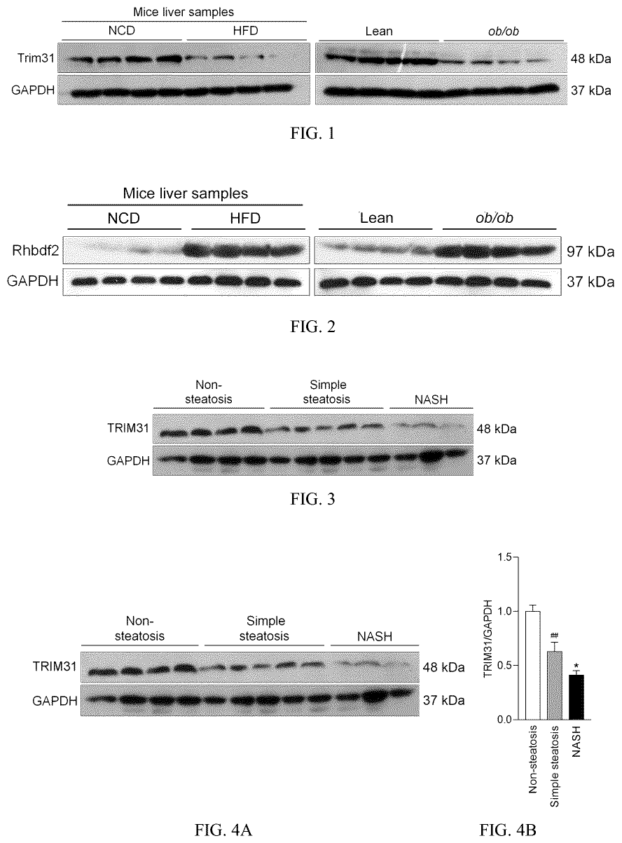

We found that Trim31 protein expression was significantly lower in liver samples of mice fed a high fat diet (HFD) for 16 weeks and ob ob mice than in mice fed a standard normal chow diet (NCD) and lean controls, accompanied by increased Rhbdf2 expression levels (FIG. 1 and FIG. 2).

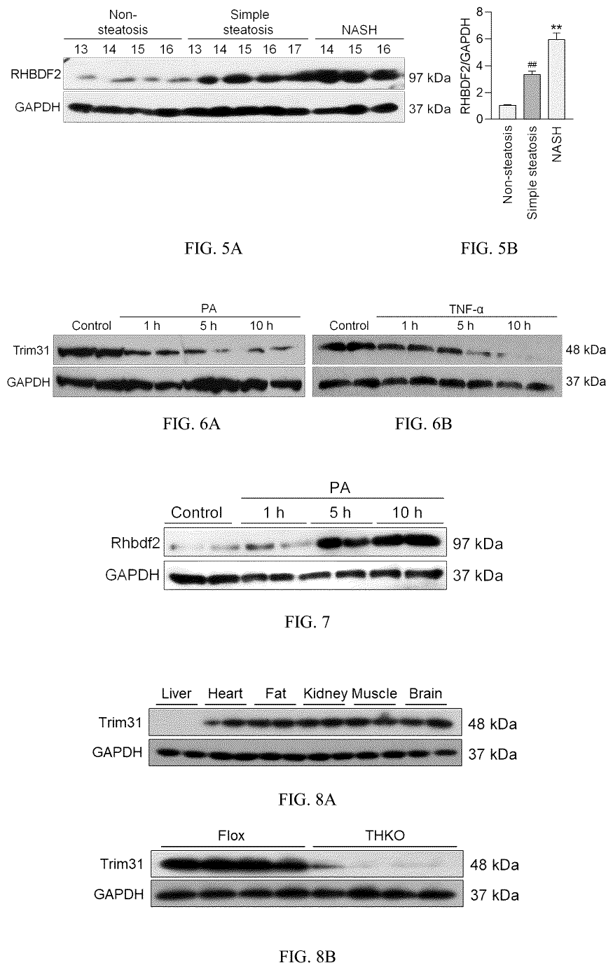

Besides, we examined its expression profile in liver samples of NAFLD and NASH patients. In patients with NAFLD or NASH, we found that Trim31 levels were drastically reduced, but hepatic Rhbdf2 levels were increased, compared to the levels in non-steatosis samples. Of note, significantly lower expression levels of TRIM31 were observed in the livers from NASH patients than than in those from patients with only with simple steatosis (FIGS. 4A-4B and FIGS. 5A-5B).

In cultured primary mouse hepatocytes, we observed that administration with palmitic acid (PA) and tumor necrosis factor-α (TNF-α) markedly reduced the Trim31 protein expression levels, accompanied by elevated Rhbdf2 expression levels (FIGS. 6A-6B, FIG. 7).

Importantly, our study also indicated that strong inactivation of Trim31 was observed in inflammation-related diseases, accompanied by a significant increase of Rhbdf2 activity. Also, Rhbdf2 has been shown to be regulated by ubiquitination modification. Consistent with these studies, we confirmed that Rhbdf2 levels were significantly increased in livers of human patients with NASH and simple steatosis phenotypes, as compared to the levels in non-steatosis samples. Meanwhile, higher expression levels of Rhbdf2 were further observed in the livers of NASH patients than in the livers of patients with only simple steatosis (FIG. 2).

### Example 2 Trim31 Ameliorates Insulin Resistance and Abnormal Glucose Metabolism

Given the tight correlation of Trim31 with fatty liver, we constructed a series of mice models to explore the role of Trim31 in regulation of the major hallmarks of insulin resistance and glycometabolic disorder. Insulin resistance and glucose metabolism disorder induced by HFD are common complications and significant inducement of fatty liver.

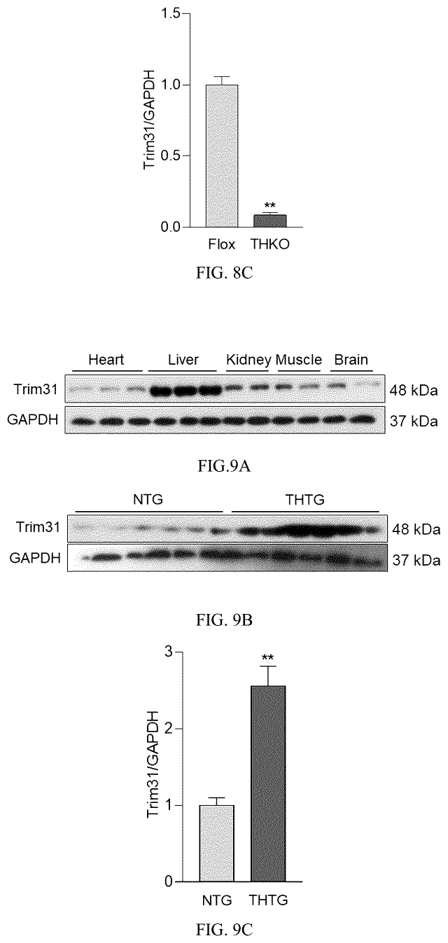

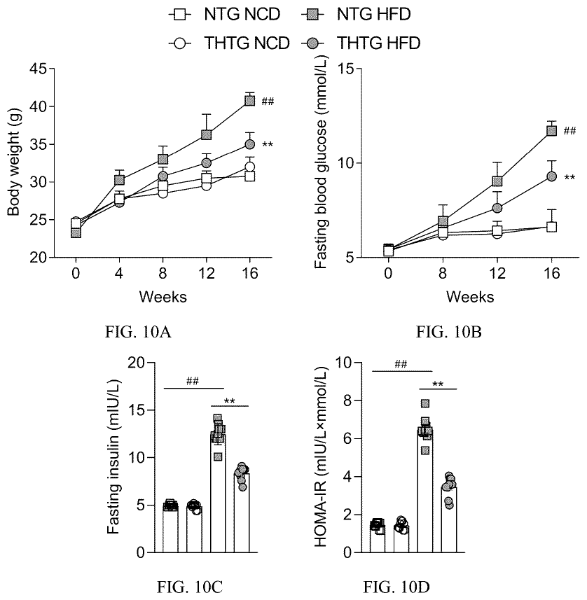

We established hepatocyte-specific Trim31 knockout (THKO) mice (FIGS. 8A-8C) and hepatocyte-specific Trim31 overexpression (THTG) mice (FIGS. 9A-9C) to examine the protective effects of Trim31 on HFD-induced insulin resistance and glucose metabolism disorder, which are common complications and vital predisposing factors of fatty liver. As expected, Trim31 dysfunction markedly increased the prolonged HFD-triggered elevations in their body weight, fasting blood glucose levels, fasting insulin levels, and HOMA-IR index, but these were alleviated by Trim31 overexpression (FIGS. 10A-10D).

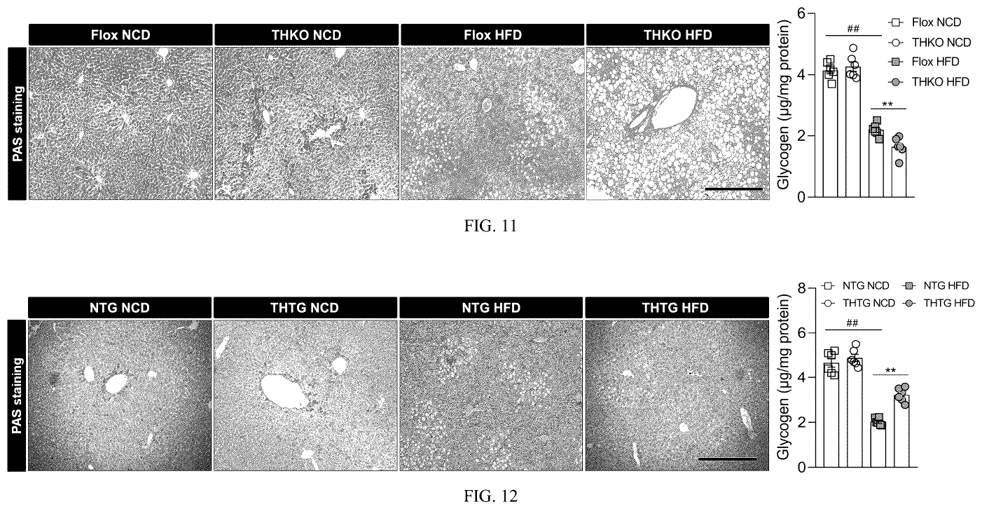

Accordingly, by using glycogen detection kit, we found that the decrease of liver glycogen reserve was significantly accelerated by Trim31 deletion (FIG. 11) but restrained by Trim31 overexpression (FIG. 12). The obtained results revealed that liver Trim31 is a crucial suppressor of HFD-triggered insulin resistance and abnormal glucose metabolism.

### Example 3 Trim31 Protects Against Liver Steatosis and Inflammation

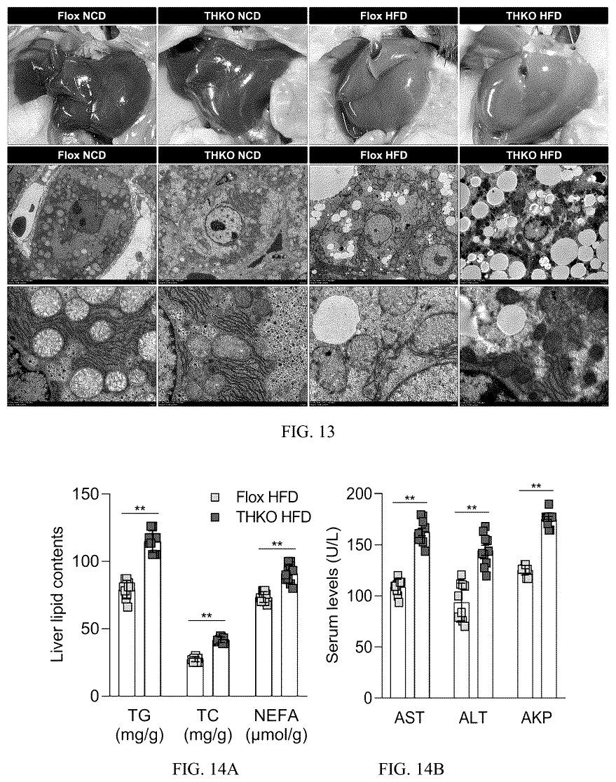

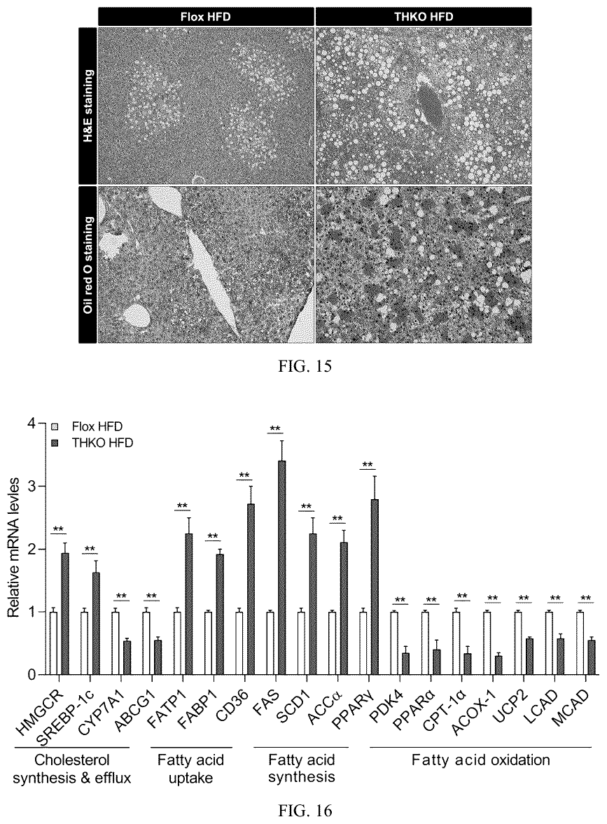

We next investigated the role of Trim31 in improving the main features of liver steatosis and inflammation. Meanwhile, the deposition of liver lipids, as determined by liver observations, transmission electron microscopy (TEM) analysis, hematoxylin and eosin (H&E) staining, Oil red O staining, and measurements of the levels of TG, total cholesterol (TC), non-esterified fatty acids (NEFA), and the liver function-related indicators serum alanine transaminase (ALT), aspartate aminotransferase (AST), and alkaline phosphatase (AKP), was visibly enhanced in HFD-treated THKO mice (FIGS. 13-15).

Functional loss of Trim31 in the liver was also involved in markedly upregulated expression of fatty acid uptake and synthesis-related genes and with dramatically downregulated expression of fatty acid β-oxidation-related genes in HFD-fed THKO mice (FIG. 16). In THTG mice, the opposite observations were made.

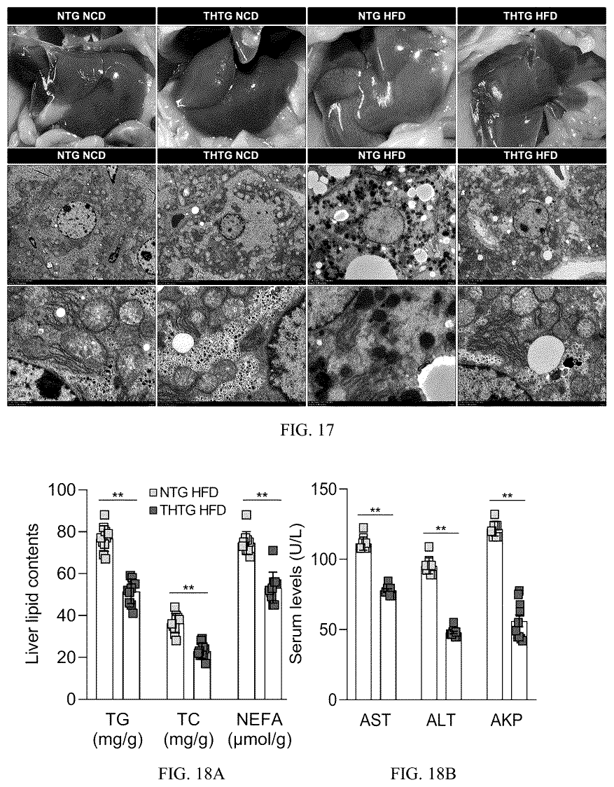

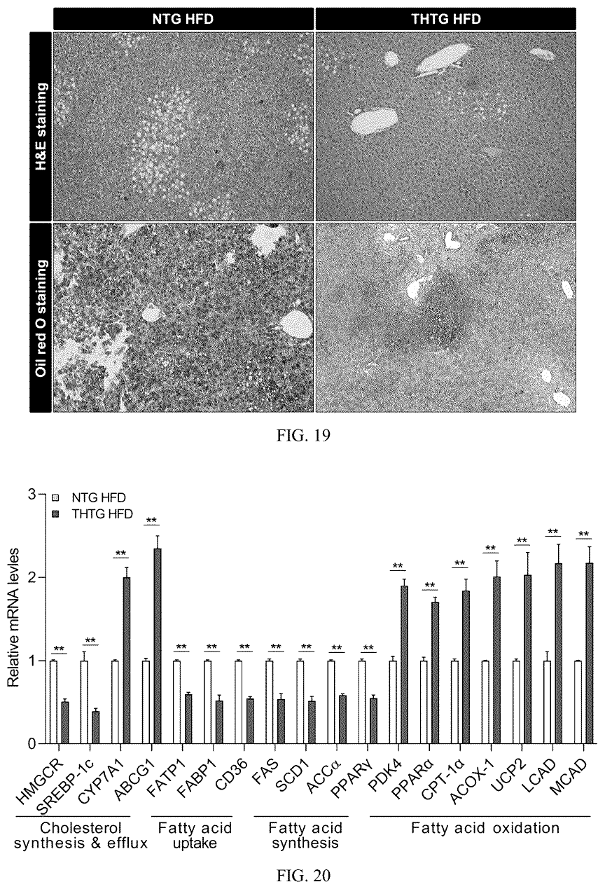

HFD-fed THTG mice displayed decreases in liver weight and in the LW/BW ratio, compared to HFD-fed non-transgenic (NTG) controls. Then, by a similar approach, we found that hepatic lipid accumulation and hepatic function-associated parameters including TG, TC, NEFA, ALT, AST and AKP were significantly reduced in HFD-fed THTG mice (FIGS. 17-19). Likewise, the expression of fatty acid uptake-and synthesis-related genes was decreased and the expression of fatty acid β-oxidation-related genes was accordingly increased in THTG mice compared to the NTG HFD group (FIG. 20).

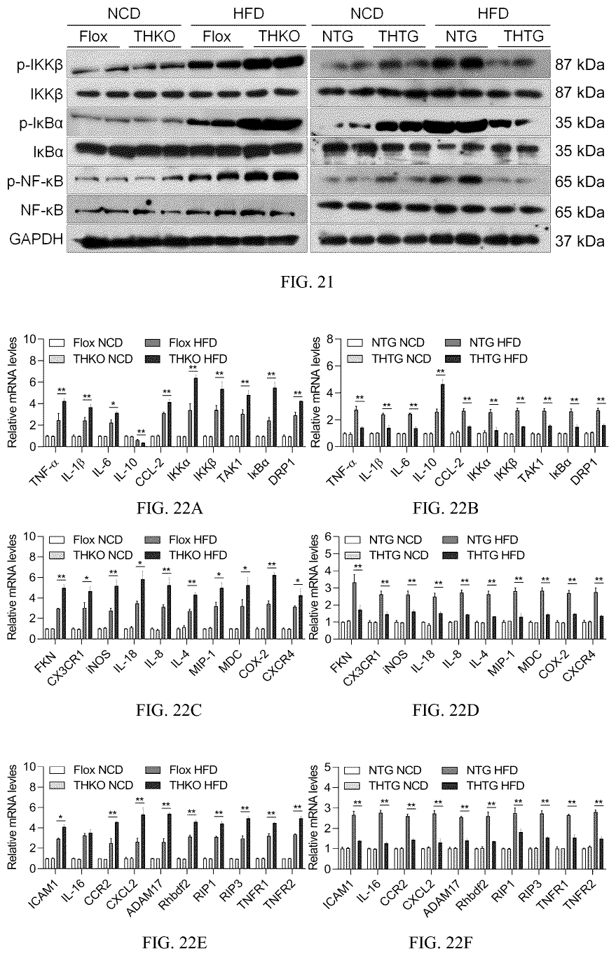

To further examine whether Trim31 participates in the regulation of liver inflammation, we investigated the activity of inflammatory signal pathways and corresponding alterations in the expression profiles of inflammation-associated cytokines and chemokines. Indeed, HFD-triggered overexcitation of inflammation-related signaling was dramatically enhanced in THKO mice, but this effect was reversed in THTG groups, as indicated by the changes in protein expression of phosphorylated IKKβ, IκBα and NF-κB, and changes in mRNA expression of inflammation-associated indicators (FIGS. 21 and 22A-22F).

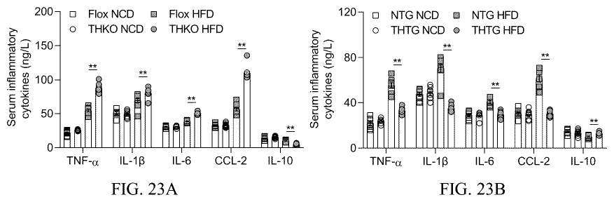

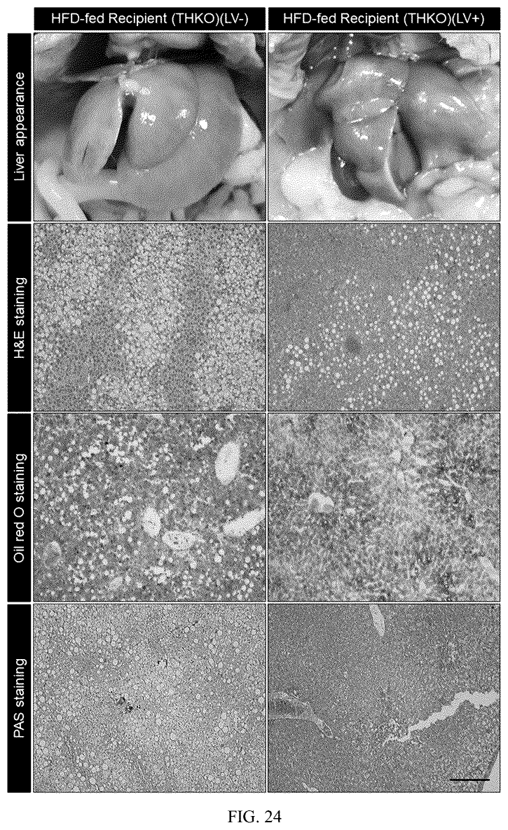

In addition, the contents of inflammatory mediators including TNF-α, IL-1β, IL-6, and CCL-2 were greatly higher in serum of THKO mice but reduced in serum of THTG mice compared with controls (FIGS. 23A-23B). The above results illustrated that liver Trim31 is an important inhibitor of HFD-triggered inflammation and liver lipid deposition in mice.

### Example 4 Trim31-Mediated Ex Vivo Gene Therapy Facilitates the Mitigation of Fatty Liver

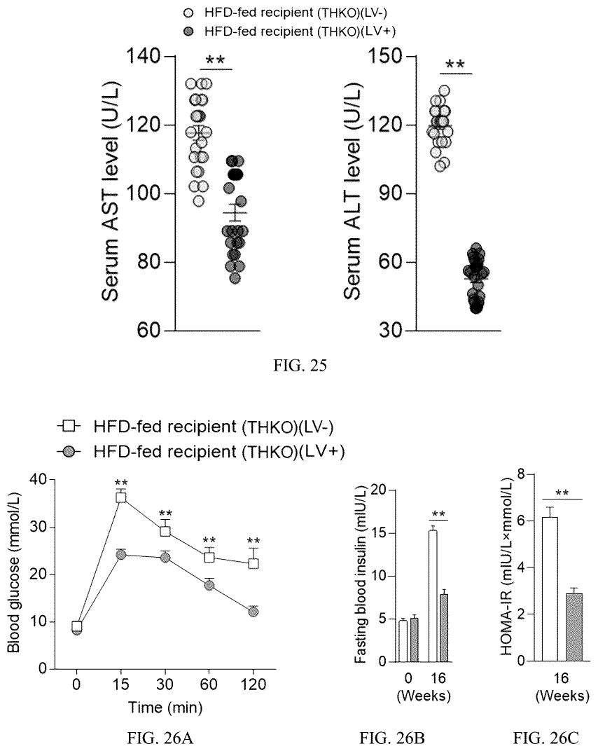

To better confirm the inhibitory effect of Trim31 on steatohepatitis, the ex vivo gene therapy intervention approach by lentivirus loaded with mouse full-length Trim31 sequences (LV-Trim31) or human full-length Trim31 sequences (LV-hTRIM31) was employed to further evaluate the role of Trim31 on mitigation of liver steatosis, insulin resistance and inflammation. Unsurprisingly, using ex vivo cultured and transduced hepatocytes, the mice with hepatocyte-specific Trim31 gain-of-function (THKO)(LV+) exhibited a remarkable decrease in HFD-induced liver weight, body weight, LW/BW ratio, AST and ALT levels, and hepatic lipid deposition, compared to the corresponding controls (THKO)(LV−) (FIGS. 24-25).

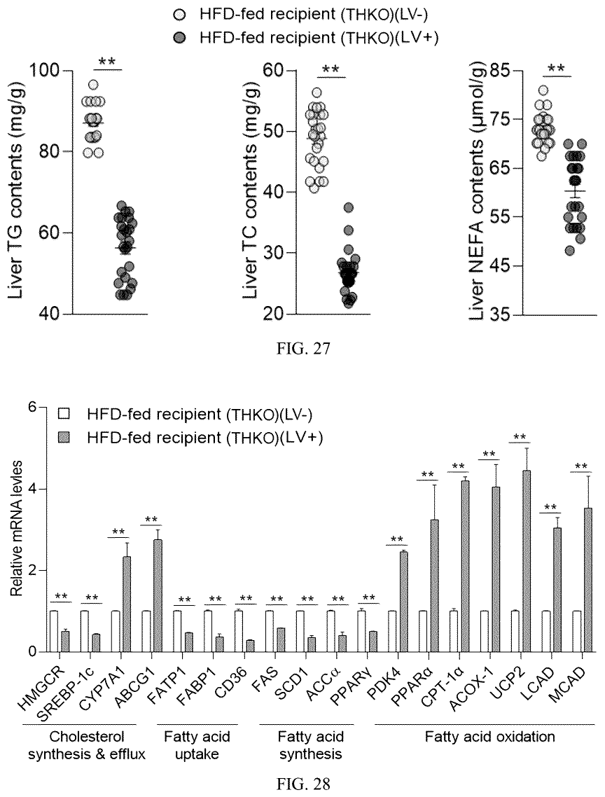

Furthermore, the THKO (LV+) mice also had lower blood glucose levels than those of THKO (LV−) controls, as confirmed by results from the GTT and ITT test (FIGS. 26A-26C). Furthermore, the high TG, TC, and NEFA contents in the liver were greatly decreased in THKO (LV+) mice (FIG. 27). Meanwhile, restoration of Trim31 expression in hepatocytes significantly reduced the expression of genes involved in fatty acid uptake and synthesis and promoted the expression of genes involved in fatty acid β-oxidation in HFD-fed THKO (LV+) mice (FIG. 28).

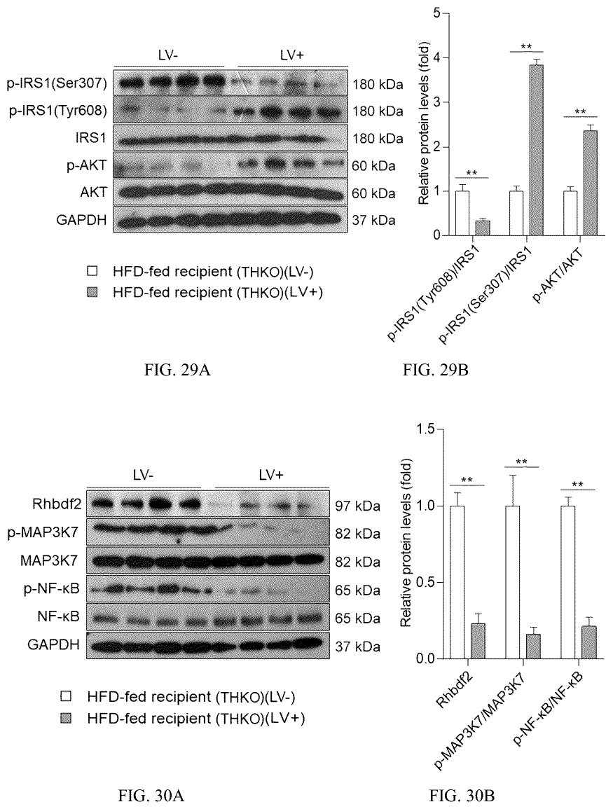

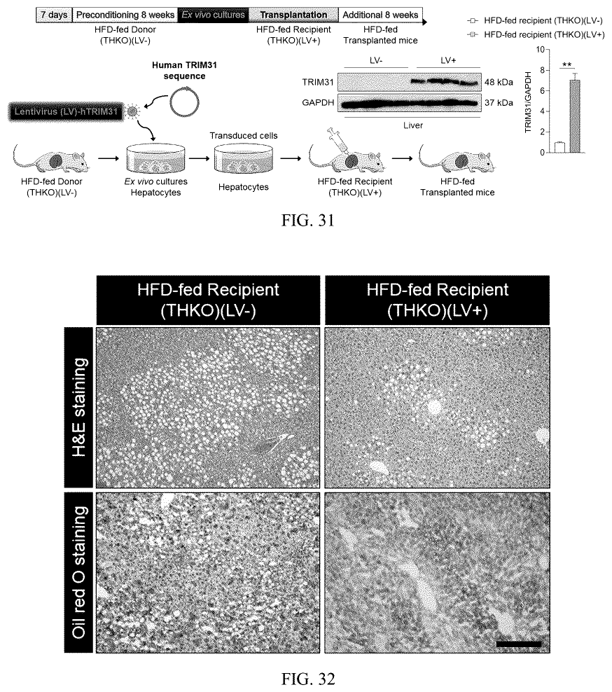

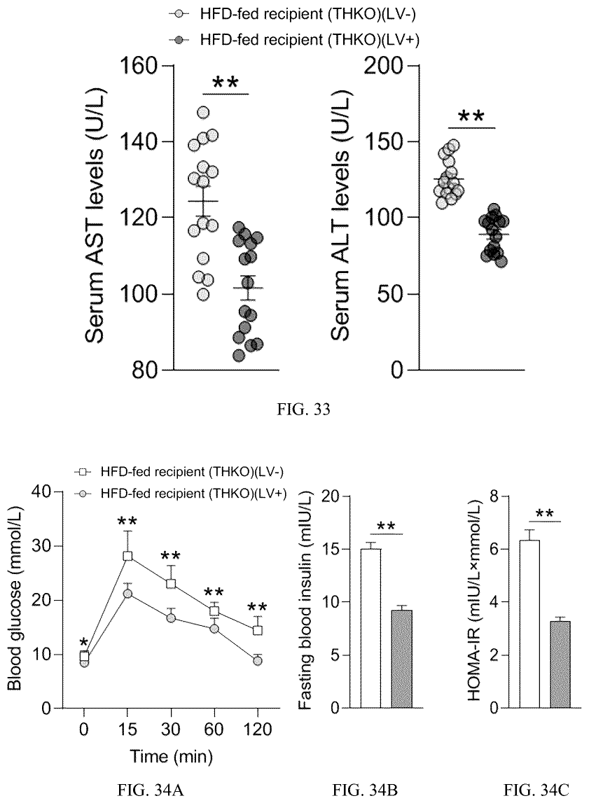

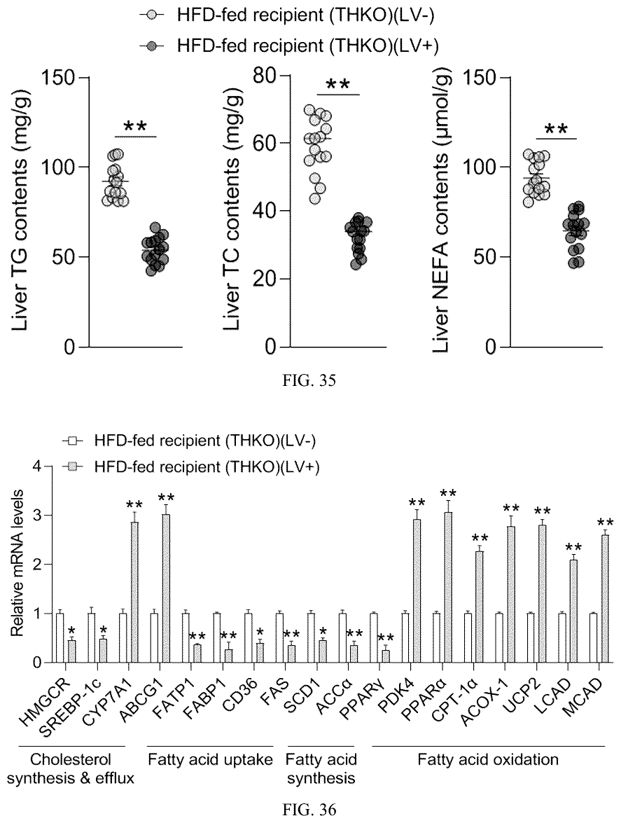

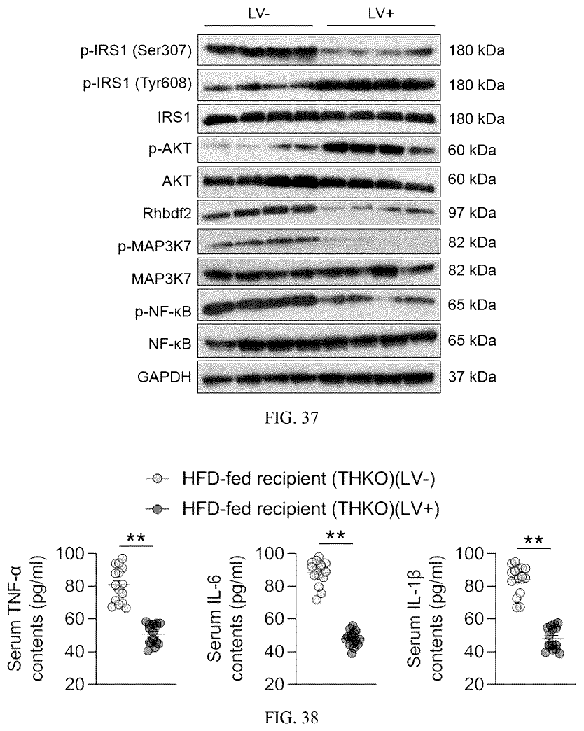

Also, the impaired insulin signaling and activated inflammation-associated signaling were markedly altered in THKO (LV+) mice compared to controls, as determined by immunoblotting, mRNA expression analysis, and measurements of the concentrations of pro-inflammatory mediators (FIGS. 29A-29B and FIGS. 30A-30B). Consistent with the above data, the ex vivo gene therapy by LV-hTRIM31 further demonstrated that hepatocytes with human TRIM31 restoration significantly alleviated HFD-induced insulin resistance, liver steatosis and inflammation (FIGS. 31-38). These results might be partly and possibly extrapolated to human pathophysiology, or provide some evidence for pathological research.

### Example 5 Inactivation of the Rhbdf2-MAP3K7 Axis by Trim31 Suppresses NAFLD Progression

Given the consistent and remarkable prohibitive function of Trim31 on fatty liver and its associated pathological phenotypes, the above results prompted us to study the molecular mechanism of Trim31 and its intrinsic function. Because Rhbdf2 is ubiquitinated, and it plays a key role in promoting development of NAFLD and NASH phenotype, we then investigated the influence of Trim31 on Rhbdf2 and its downstream events components.

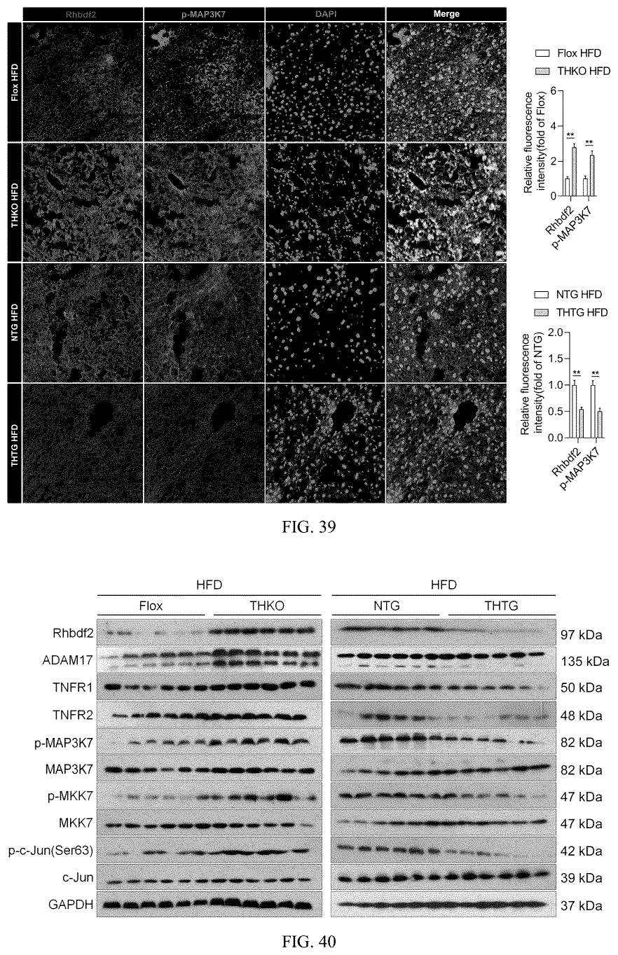

As expected, significant activation of Rhbdf2-MAP3K7 axis induced by HFD treatment was boosted in the THKO mice, but was greatly repressed in the THTG mice, as indicated in immunofluorescence analysis of tissue sections (FIG. 39). Likewise, Rhbdf2-MAP3K7 signaling members including ADAM17, TNFR1/2, MKK7 and c-Jun, were also determined to be involved in fatty liver progression. The levels of ADAM17, TNFR1/2, and p-MAP3K7 and its downstream component MKK7 were upregulated by Trim31 ablation but markedly inhibited by Trim31 overexpression in vivo and in vitro (FIG. 40).

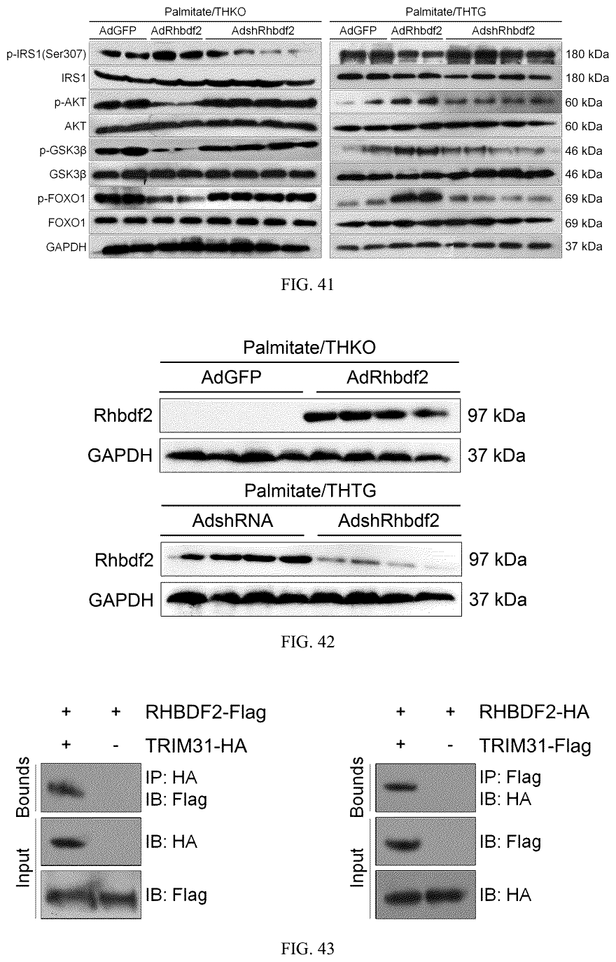

To further examine the regulatory effect of Trim31 on the function of Rhbdf2, we next constructed an impaired insulin signaling model in PA-induced isolated primary hepatocytes. The adenovirus-packed full-length Rhbdf2 sequence (AdRhbdf2) and shRNA targeting Rhbdf2 (AdshRhbdf2) were used to overexpress and inactivate Rhbdf2, respectively, in Trim31-specific deletion or Trim31-transgenic hepatocytes. Indeed, impairment of insulin signaling stimulated by PA was significantly deteriorated in AdRhbdf2-transfected THKO-hepatocytes but was virtually assuaged in AdshRhbdf2-transfected THKO-hepatocytes, as compared to controls. In contrast to this, however, the THTG-hepatocytes transfected with AdshRhbdf2 showed a decreasing trend in PA-induced impaired insulin signaling (i.e., alteration of p-AKT, p-GSK3β and p-FOXO1 levels) compared with AdRhbdf2-transfected THTG-hepatocytes (FIG. 41 and FIG. 42). The above data further demonstrated that dysfunction of the Rhbdf2-MAP3K7 signaling pathway is essential for the protective effect of Trim31 against hepatic steatosis.

### Example 6 Trim31 Directly Interacts With Rhbdf2

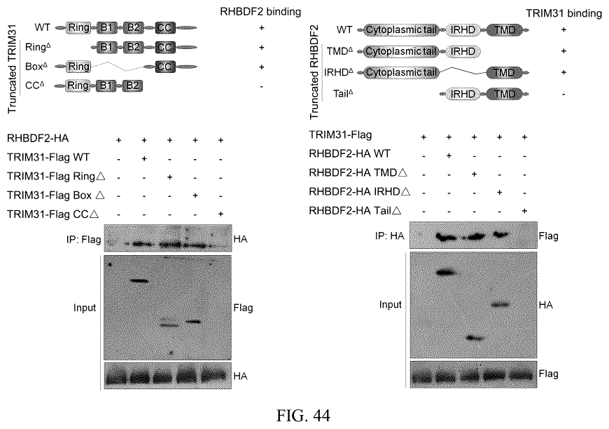

The observed effects of Trim31 on Rhbdf2 signaling-associated hepatic steatosis, inflammation, and insulin resistance prompted us to examine whether Trim31 directly interacts with Rhbdf2 during the development of NAFLD. To answer this question, an immunoprecipitation assay was employed. In vitro interaction tests suggested that exogenically expressed TRIM31 could directly bind to RHBDF2 and vice versa (FIG. 43).

Importantly, as a key member of the E3 ubiquitin ligase family, TRIM31 is mainly composed of three parts: an N-terminal RING-finger domain, a B-Box domain, and a C-terminal coiled-coil (CC) domain. To confirm which domain of TRIM31 is responsible for the interaction with RHBDF2, a series of vectors encoding Flag-tagged truncated TRIM31 mutants, including wild-type (TRIM31-Flag WT), a RING-finger domain ablation mutant (TRIM31-Flag RING^{Δ}), a B-Box domain deletion mutant (TRIM31-Flag Box^{Δ}), and a CC domain deletion mutant (TRIM31-Flag CC1), were generated for the following binding experiments.

The co-immunoprecipitation assays demonstrated that RHBDF2 co-precipitated with TRIM31 WT, TRIM31 RING^{Δ}, and TRIM31 Box^{Δ}. These results indicated that the CC domain contributes to binding to RHBDF2. The N-terminal cytoplasmic tail (Tail) domain of RHBDF2 is essential for the interaction with TRIM31 (FIG. 44).

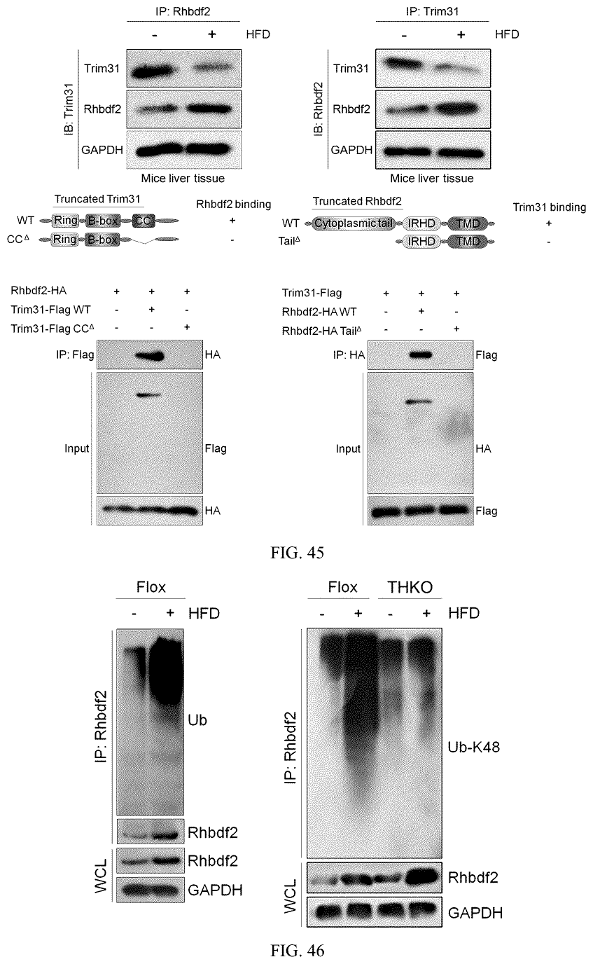

In addition, because the CC domain of TRIM31 and the N-terminal (Tail) domain of RHBDF2 in human are essential for their interaction, we examined whether they have a similar biological function in rodent. Thus, the binding domains of mouse Trim31 and Rhbdf2 were also determined using Trim31-Flag CC^{Δ}, Rhbdf2-HA Tail^{Δ}, and their corresponding wild-type vectors. Consistent with the protein binding results, in transfected mice hepatocytes, Trim31 with CC domain deletion did not bind to Rhbdf2 (FIG. 45).

### Example 7 Trim31 Facilitates Degradation of Rhbdf2 via K48-Linked Polyubiquitination

In the Embodiments of the invention, it was verified that Rhbdf2 could be regulated by Trim31 via its E3 ubiquitin ligase activity, consistent with previous speculation that the cytoplasmic domain of Rhbdf2 may contain sites for K48-polyubiquitination, and therefore proteasomal degradation. In example 6, Trim31 have confirmed to be a Rhbdf2-related protein (FIG. 44). Indeed, a significant increase in Rhbdf2 ubiquitination levels was observed in vitro and in vivo (FIG. 46).

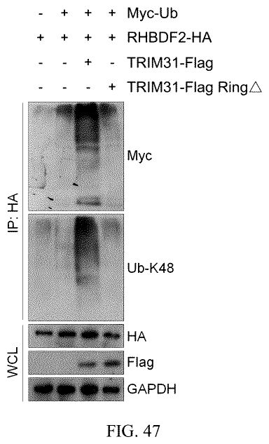

Also, Rhbdf2 was co-transfected with Myc-ubiquitin and Flag-tagged WT Trim31 into L02 cells. Rhbdf2 ubiquitination levels were greatly enhanced in the presence of a Trim31 expression vector. Of note, the Trim31 expression vector with RING-finger domain ablation mutant fails to catalyze ubiquitination of Rhbdf2, suggesting the RING-finger domain is required for Trim31 function in the regulation of Rhbdf2 ubiquitination (FIG. 47).

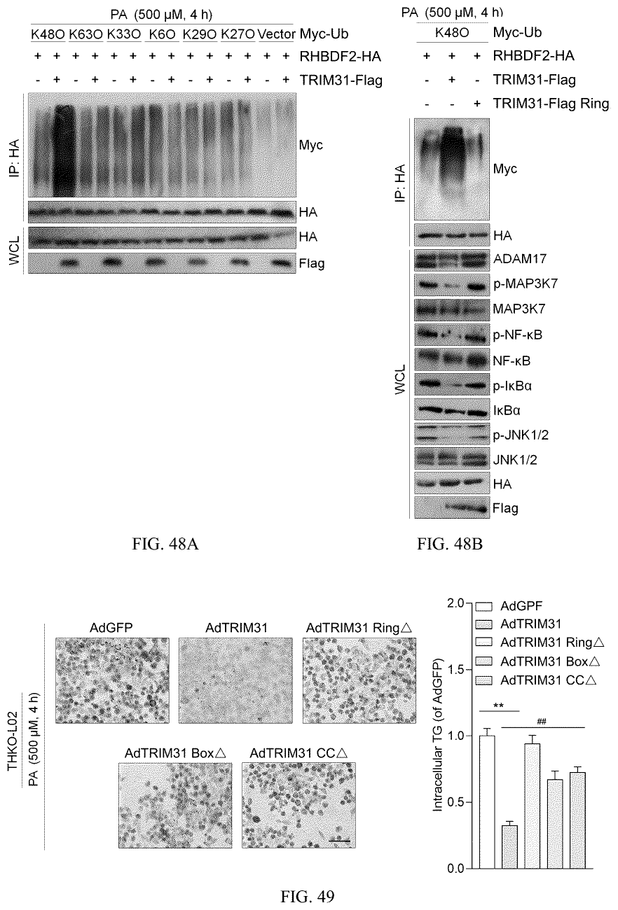

Furthermore, to accurately investigate the manner of Trim31-regulated Rhbdf2 polyubiquitination, the ubiquitin mutation plasmids K48, K63, K33, K6, K29 and K27 were used for in vitro transfection experiments. The label of “O” in K48O, K63O, K33O, K6O, K29O and K27O reveal ubiquitin in which all lysine residues except themselves were completely mutated. Rather, Trim31-mediated increase in polyubiquitination of Rhbdf2 could be greatly observed in the presence of K48-loading vector, but not with other vectors (FIGS. 48A-48B).

It is generally considered that the RING-finger domain mutant of Trim31 has virtually lost its E3 ubiquitin ligase activity. Given the essential role of the RING-finger domain in Trim31 function, indeed, deletion of RING-finger domain of Trim31 not only abolished the polyubiquitination of Rhbdf2, but also impeded the ability of Trim31 to restrain the activation of the Rhbdf2-MAP3K7 pathway and its downstream signaling cascades (e.g., ADAM17, p-NF-κB, p-IκBα and p-JNK1/2) (FIGS. 48A-48B).

The adverse effect of RING-finger domain deficiency on Trim31 function was further supported by the changes in intracellular TG concentrations in PA-stimulated cells in a series of vector-transfected Trim31-deficient L02 cells (THKO-L02) (FIG. 49).

The above data indicated that Trim31-induced degradation of Rhbdf2 by K48-linked polyubiquitination depends on its E3 ubiquitin ligase activity.

### Example 8 Trim31-Rhbdf2 Interaction is Required for Trim31-Regulated Hepatic Steatosis and Inflammation

Given the strong correlation of Rhbdf2 activity with Trim31 E3 ubiquitin ligase activity, to thoroughly explore whether the Trim31-Rhbdf2 interaction is required for the protective function of Trim31 on hepatic steatosis, a lentivirus loaded with Trim31 with a RING-finger domain ablation (LV-Trim31 RING^{Δ}) was generated and used to further investigate the influence of Trim31 RING^{Δ} on HFD-induced liver steatosis, insulin resistance, and inflammation. As expected, in the ex vivo experiment, in HFD-fed mice, Trim31 RING^{Δ} hepatocyte transplantation (THKO)(LV+) failed to alter liver weight, body weight, the LW/BW ratio, AST and ALT levels, and liver lipid accumulation, compared to controls (THKO)(LV−). Additionally, analysis of blood glucose levels and contents of hepatic TG, TC and NEFA indicated no significant differences.

Additionally, analysis of blood glucose levels and contents of hepatic TG, TC and NEFA indicated no significant differences. Furthermore, no significant changes in insulin signaling activity or in the Rhbdf2-MAP3K7 axis and downstream events were observed compared to controls, as revealed by immunoblotting, mRNA quantification, and measurements of pro-inflammatory mediators.

Consistent with these findings, the ex vivo gene therapy by LV-hTRIM31 further demonstrated that mice transplanted with human TRIM31 RING^{Δ}-hepatocytes transplantation also did not markedly alleviate HFD-induced insulin resistance, liver steatosis and inflammation These data further indicated that the RING domain of Trim31 in mice and TRIM31 in human is essential for the protective function of E3 ubiquitin-protein ligase.

Collectively, the Trim31-Rhbdf2 interaction and the RING-finger domain of Trim31 are essential for the effects of Trim31 on regulation of the hepatic steatosis and inflammation.

### Example 9 Targeting Rhbdf2 is Required for the Protective Effects of Trim31 Against Hepatic Steatosis

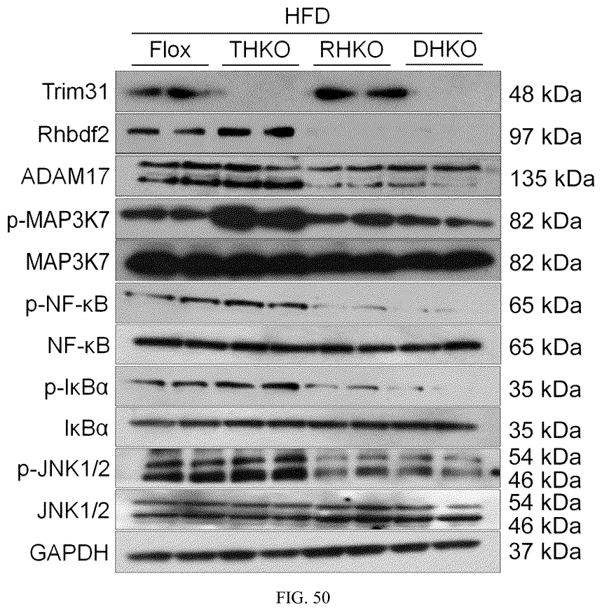

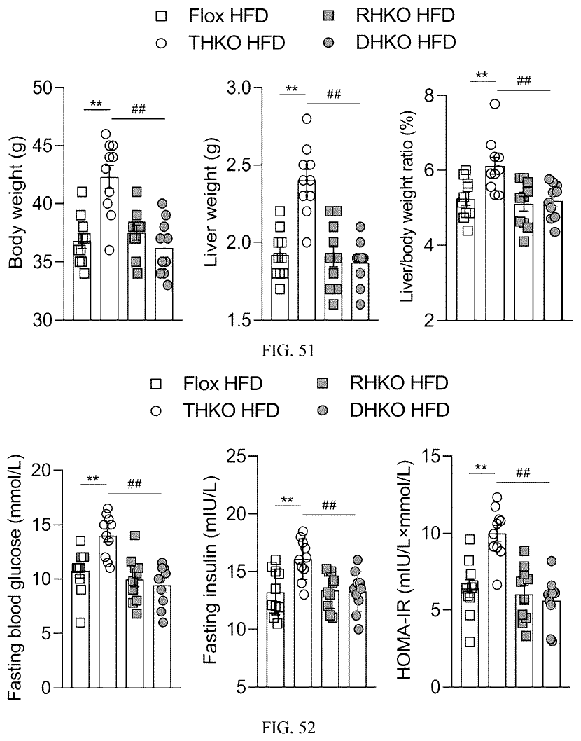

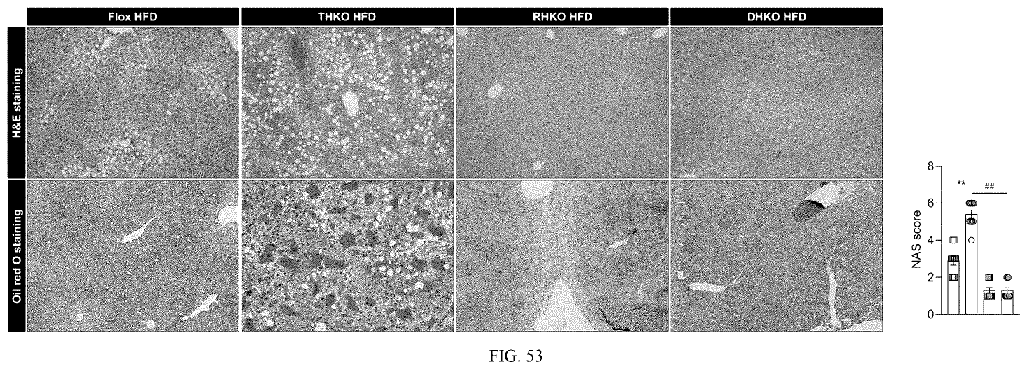

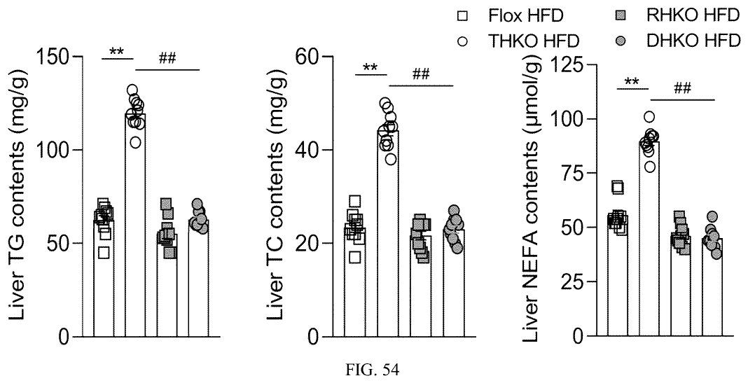

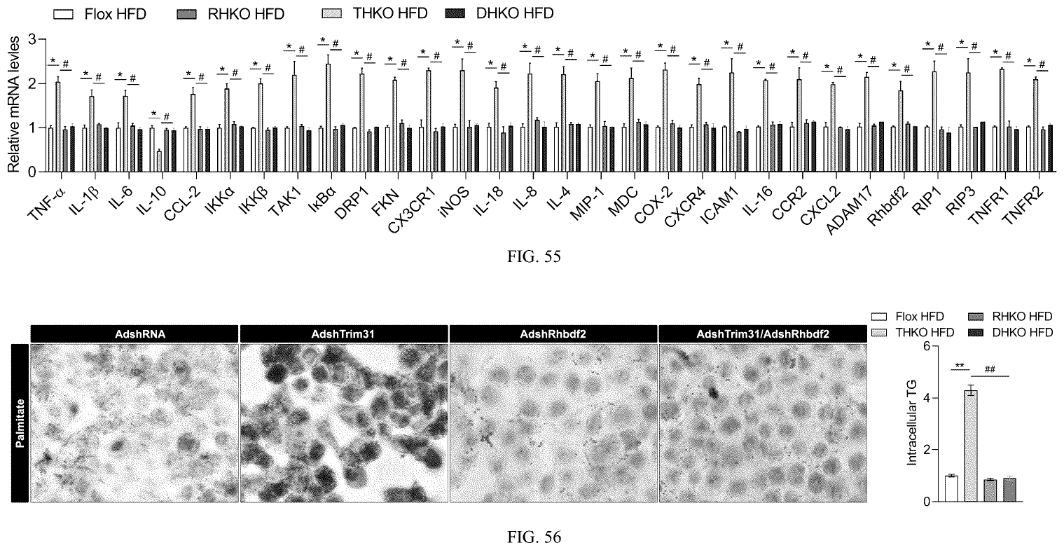

To further confirm that the inhibition of Rhbdf2 signal mediates the protective function of Trim31 against fatty liver, the mice with hepatocyte-specific Trim31 deficiency (THKO) mice were mated with Rhbdf2^{flox/flox} mice (Alb-Cre; Rhbdf2^{flox/flox,} hereafter referred to as RHKO), as indicated in the Materials and Methods section, to generate hepatocyte-specific Trim31 and Rhbdf2 double deletion mice (Alb-Cre; Rhbdf2^{flox/flox}, Trim 31^{flox/flox}, hereafter referred to as DHKO). Both of Trim31 and Rhbdf2 deficiency in liver samples was determined by western blotting analysis (FIG. 50). Deficiency of Rhbdf2-triggered mitigation of prolonged HFD-induced steatohepatitis has been intrinsically confirmed by our previous study. As expected, indeed, Rhbdf2 deletion blocked the effects of Trim31 ablation on the HFD-stimulated upregulation of the Rhbdf2-MAP3K7 axis and downstream signaling cascades, body weight, liver weight, LW/BW ratio, insulin signaling, blood glucose levels, liver lipid deposition, and elevated expression of pro-inflammatory genes (FIGS. 51-55). In addition, PA-induced changes in intracellular TG levels in vitro also showed the same trend (FIG. 56). These results indicated that the Rhbdf2 pathway is required for the protective effects of Trim31 against NAFLD.

### Example 10 Evaluation of Therapeutic Feasibility and Effect of Targeting Trim31-Rhbdf2 Administered Signaling in Nonalcoholic Steatohepatitis (NASH)

Based on the effective protective effect of Trim31 in NAFLD, we accordingly investigated the therapeutic feasibility and effect of targeting the Trim31-Rhbdf2-MAP3K7 pathway in the development and progression of NASH. The THTG mice were then fed with an HFHF diet containing 14% protein, 42% fat, 44% carbohydrates, and 0.2% cholesterol and with a total of 42 g/L of carbohydrate mixed in drinking water at a ratio of 55% fructose and 45% sucrose by weight, for 16 weeks to generate mice with a NASH phenotype (THTG-HFHF).

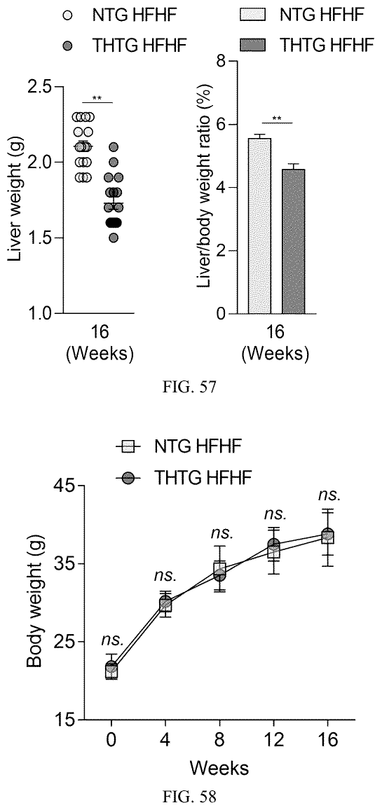

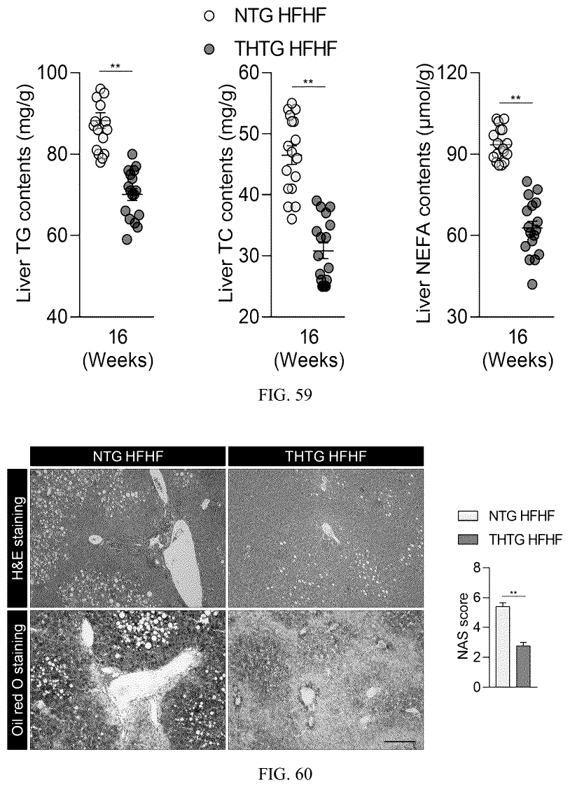

NTG mice with ad libitum access to this diet for 16 weeks were used as controls (NTG-HFHF). Unsurprisingly, in the HFHF-stimulated NASH model, the liver weight, LW/BW ratio and concentrations of hepatic TG, TC and NEFA levels were markedly lower in the THTG group than in the NTG controls after 16 weeks on an HFHF diet, accompanied by no significant difference in body weight between the THTG-HFHF and NTG-HFHF mice (FIGS. 57-59).

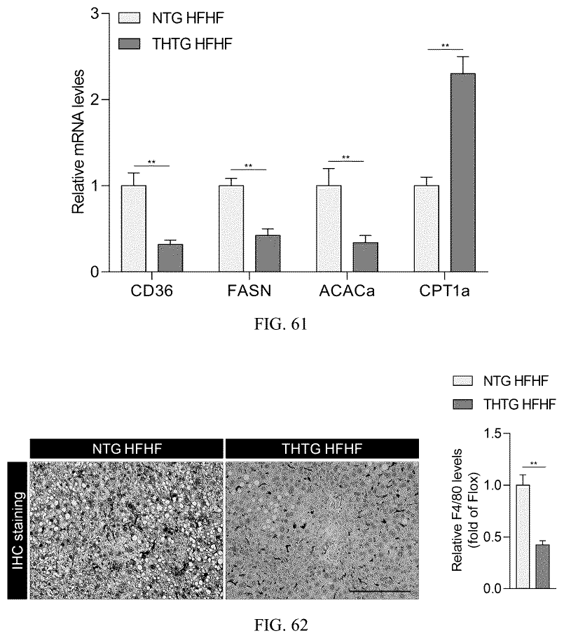

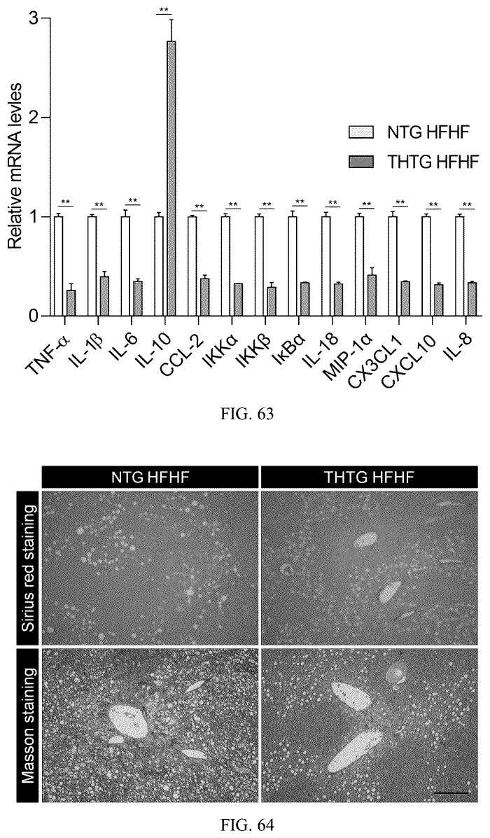

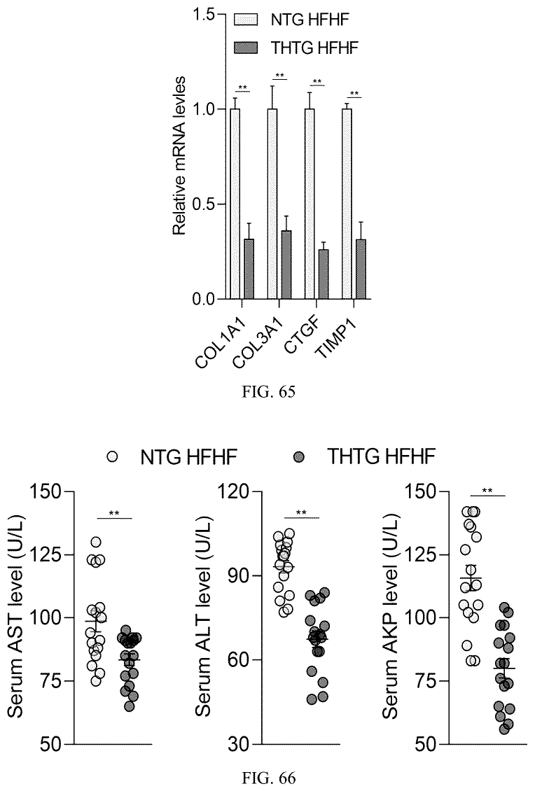

Also, compared to NTG mice, remarkable decreases in liver lipid deposition, expression of fatty acid synthesis genes (e.g., CD36, FASN, and ACACa), expression of inflammation-related genes (e.g., TNF-α, IL-1β, IL-6, and CCL-2), liver fibrosis (i.e., collagen deposition), and expression of collagen synthesis-related genes (e.g., TIMP1, CTGF, COL1A1, and COL3A1) were observed in THTG mice after HFHF diet treatment (FIGS. 60-65), as determined by H&E staining, Oil red O staining, F4/80 immunohistochemical assay, Sirius red staining, Masson staining, and measurements of the expression levels of inflammation-and collagen-associated genes. Moreover, Trim31 overexpression also decidedly downregulated mice serum AST, ALT and AKP levels, pro-inflammatory cytokines TNF-α, IL-6 and IL-1β concentrations (FIG. 63), and the Trim31-regulated Rhbdf2-MAP3K7 axis and downstream signaling cascades after 16 weeks of HFHF challenge. Collectively, our data revealed the positive effects of Trim31 on mitigation of steatohepatitis and associated metabolic syndrome in mice.

In the embodiment of the invention, hepatocyte-specific TRIM31 knockout mice (THKO)/transgenic mice (THTG) and lentivirus-mediated ex vivo gene therapy (LV-TRIM31) mice were used to identify hepatic TRIM31 as a significant negative regulator of high-fat diet (HFD/HFHF)-induced or genetically induced chronic inflammation, insulin resistance, hepatic steatosis, and NASH. The ex vivo gene therapy including but not limited to lentiviral-TRIM31 (LV-TRIM31) and Adeno-associated-viral-TRIM31 (AAV-Trim31) could significantly alleviate the pathological phenotype of NASH and has the potential to treat NASH.

### Example 11 Betacyanin (BCN) Alleviates Liver Fibrosis and Inflammation in Mice by Activating TRIM31

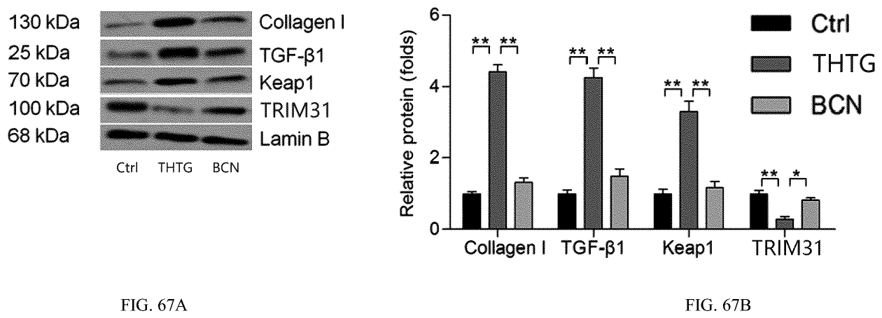

Mice with a NASH phenotype (THTG-HFHF) were constructed according to the protocol described in Example 10. A control group (Ctrl), a NASH group (THTG), and a drug group (BCN) were set up. The drug group was fed with 0.2 g/kg betacyanin (BCN) mixed with feed. As shown in FIGS. 67A-67B, after 16 weeks, the expression levels of Collagen I, TGF-β1 and Keap1 proteins were significantly increased, and the expression of TRIM31 protein was significantly decreased in the liver of THTG-HFHF mice, indicating that high glucose and lipid caused collagen accumulation and oxidative stress leading to non-alcoholic steatohepatitis. BCN treatment significantly reduced the expression levels of Collagen I, TGF-β1 and Keap1 proteins and increased the expression of TRIM31 protein, which was close to the normal level of the control group, and effectively alleviated liver fibrosis and inflammation.

### Example 12 Fisetin (FIS) Alleviates Liver Fibrosis and Inflammation in Mice by Activating TRIM31

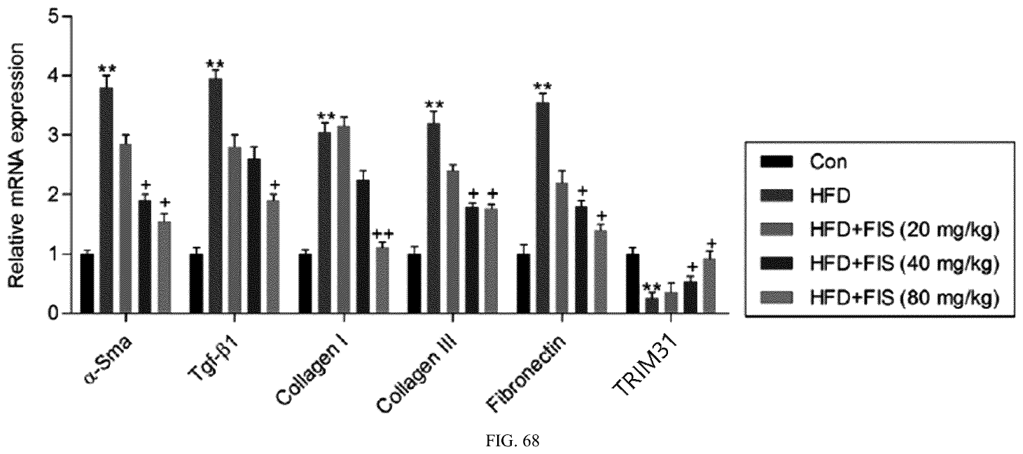

Mice with a NASH phenotype (THTG-HFHF) were constructed according to the protocol described in Example 10. A control group (Ctrl), a NASH group (THTG), and three drug groups (FIS) were set up. The three drug groups (FIS) were treated with 20 mg/kg, 40 mg/kg and 80 mg/kg fisetin (FIS) by gavage. As shown in FIG. 68, after 16 weeks, the mRNA levels of α-SMA, Collagen I, TGF-β1 and Fibronectin were significantly increased, and the mRNA level of TRIM31 protein gene was significantly decreased in the liver of THTG-HFHF mice. These results indicate that the accumulation of collagen and oxidative stress caused by high glucose and lipid may lead to non-alcoholic steatohepatitis. The mRNA levels of α-SMA, Collagen I, TGF-β1 and Fibronectin in the liver tissues of mice treated with low, medium and high doses of fisetin were significantly decreased, and the mRNA level of TRIM31 protein was significantly increased. Especially, in the high dose group, these indexes were close to the normal level of the control group, indicating that fisetin significantly alleviated liver fibrosis and inflammation.

### Example 13 Carminic Acid (CA) Alleviates Liver Fibrosis and Inflammation in Mice by Activating TRIM31

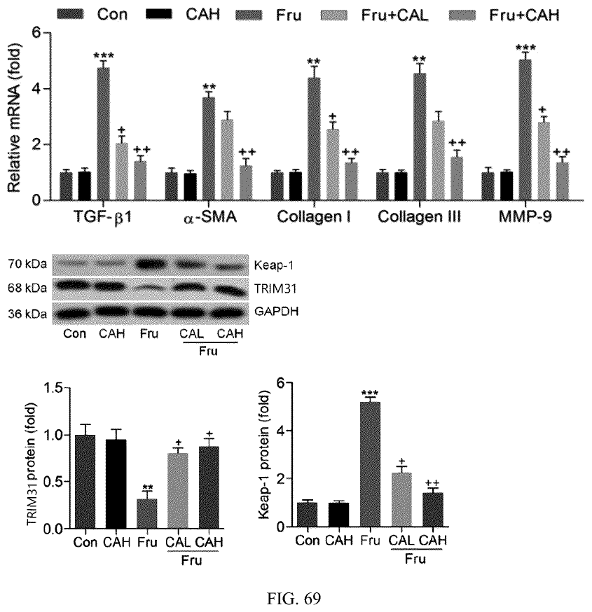

Mice with a NASH phenotype (THTG-HFHF) were constructed according to the protocol described in Example 10. The control group (Ctrl), NASH-THTG group (Fru), low dose group (CAL, 0.5% CA+Fru), high dose group (CAH, 1% CA+Fru) were set up. The drug group was treated with 0.5% and 1% carminic acid (CA) by gavage. As shown in FIG. 69, after 16 weeks, the mRNA levels of a-SMA, Collagen I, TGF-β1, MMP-9 were significantly increased, the protein expression level of Keap1 was significantly increased, and the protein expression of TRIM31 was significantly decreased in the liver of THTG-HFHF mice. These results indicate that the accumulation of collagen and oxidative stress caused by high glucose and lipid may lead to non-alcoholic steatohepatitis. After treated with low and high doses of CA, the mRNA levels of α-SMA, Collagen I, TGF-β1, MMP-9 protein genes and Keap1 protein were significantly decreased and the protein expression of TRIM31 was significantly increased in the liver tissues of mice. Especially, in the high dose group, these indexes were close to the normal level of the control group, indicating that CA could effectively alleviated liver fibrosis and inflammation.

### Example 14 Small Molecule Compounds Promote TRIM31 mRNA Expression Levels

Palmitic acid/oleic acid (PA/OA) was used to induce the decrease of TRIM31 mRNA expression in human hepatocyte L02 cells. The cells were divided into control group and drug group. The drug group was treated with a concentration of 100 μmol/L compound, and the expression level of TRIM31 mRNA was detected by RT-qPCR. The expression level of TRIM31 mRNA in the drug group divided by that in the control group (Q_{drug}/Q_{ctrl}) was used to evaluate the ability of the compounds to promote TRIM31 expression. As shown in Table 1, the mRNA expression level of TRIM31 downregulated by PA/OA was increased by 1.15-3.74 fold after drugs treatment.

TABLE 1
Effect of small molecule compounds on expression
levels of TRIM31 mRNA induced by PA/OA

| Compounds | Q_{drug}/Q_{ctrl} | Compounds | Q_{drug}/Q_{ctrl} |
|---|---|---|---|
| Berberine | 1.32 | Rhynchophylline | 1.92 |
| Magnoflorine | 1.38 | Camptothecin | 2.13 |
| Peganine | 1.34 | Tetrahydropalmatine | 1.76 |
| Serpentine | 1.33 | Jateorhizine | 1.72 |
| Leonurine | 1.51 | Chelidonine | 1.53 |
| Tetrandrine | 1.56 | Lycorine | 2.26 |
| Orixine | 1.69 | Acronycine | 1.43 |
| Peimine | 1.31 | Sophocarpidine | 1.30 |
| Colchicine | 1.32 | Oxymatrine | 1.15 |
| Piperine | 1.80 | Juglanin | 2.95 |
| Quercetin | 3.38 | Anthocyanidin | 3.68 |
| Dihydroquercetin | 3.32 | Proanthocyanidin | 3.12 |
| Luteolin | 3.09 | Daidzein | 2.66 |
| Morin | 2.86 | Apigenin | 3.21 |
| Mulberrin | 3.74 | Baicalein | 3.53 |
| Apiin | 3.22 | Scutellarein | 3.20 |
| Diosmetin | 3.41 | Chrysin | 2.89 |
| Icariin | 3.33 | Wogonin | 3.05 |
| Tricin | 2.90 | Cyanidin | 2.97 |
| Puerarin | 2.72 | Silymarin | 2.85 |
| Silibinin | 3.12 | Liquiritigenin | 2.79 |
| Catechin | 3.21 | Epicatechin | 2.92 |
| Genistein | 2.73 | (−)-epigallocatechin | 2.71 |
| Kaempferol | 3.26 | Galangin | 2.81 |
| Fisetin | 3.27 | Delphinidin | 2.49 |
| Isorhamnetin | 2.77 | Pelargonidin | 2.78 |
| Myricetin | 3.25 | Breviscapine | 3.29 |
| Hesperetin | 3.06 | Afzelechin | 3.12 |
| Naringenin | 3.02 | Rutin | 2.86 |
| luteolin 7-O-β-D- | 2.83 | Curcumin | 2.83 |
| glucoside | | | |
| Baicalin | 2.98 | Ferulic acid | 2.51 |
| Chlorogenic acid | 2.42 | Tea polyphenols | 2.71 |
| Cynarin | 2.56 | Cynaropicrin | 3.35 |
| Cynarascoloside C | 2.34 | Resveratrol | 3.66 |
| Punicosides | 2.64 | Lenalidomide | 1.43 |
| Penicillamine | 1.72 | Thalidomide | 1.88 |
| Pomalidomide | 1.49 | Bifendate | 1.76 |
| Glycyrrhizinic acid | 1.86 | Sorafenib | 2.86 |
| Glycyrrhizic Acid | 1.72 | Regorafenib | 2.79 |
| Monoammonium Salt | | | |
| Ademetionine | 2.55 | Lenvatinib | 2.75 |
| Ursodesoxycholic acid | 2.85 | Betacyanin | 3.14 |
| Tauroursodeoxycholic | 2.77 | Metformin | 2.45 |
| acid | | | |
| Chenodeoxycholic acid | 2.54 | Carminic acid | 2.78 |
| Taurochenodeoxycholic | 2.51 | Bicyclol | 2.45 |
| acid | | | |

### Example 15 The Extracts of Traditional Chinese Medicine Promoted the Detection of TRIM31 mRNA Expression Level

Palmitic acid/oleic acid (PA/OA) was used to induce the decrease of TRIM31 mRNA expression in human hepatocyte L02 cells. The cells were divided into control group and drug group. The drug group was treated with a concentration of 100 mg/L ethanol extracts of traditional Chinese medicine, and the expression level of TRIM31 mRNA was detected by RT-qPCR. The expression level of TRIM31 mRNA in the drug group divided by that in the control group (Q_{drug}/Q_{ctrl}) was used to evaluate the ability of the compounds to promote TRIM31 expression. As shown in Table 2, the mRNA expression level of TRIM31 downregulated by PA/OA was increased by 1.23-2.83 fold after drugs treatment.

TABLE 2
Effect of extracts of traditional Chinese medicine on
expression levels of TRIM31 mRNA induced by PA/OA

| extracts of traditional | | extracts of traditional | |
|---|---|---|---|
| Chinese medicine | Q_{drug}/Q_{ctrl} | Chinese medicine | Q_{drug}/Q_{ctrl} |
| Astragalus | 1.31 | artichoke | 2.35 |
| membranaceus | | | |
| Codonopsis pilosula | 1.42 | Thistle | 2.16 |
| Salvia miltiorrhiza | 1.77 | milk thistle | 2.28 |
| Angelica sinensis | 1.61 | Pueraria lobata | 2.11 |
| Chinese yam | 1.23 | Coptis chinensis | 2.01 |
| pseudo-ginseng | 1.64 | Phellodendron | 1.78 |
| | | amurense | |
| Schisandra chinensis | 1.75 | turmeric | 2.83 |
| Polygonatum | 1.56 | honeysuckle | 2.21 |
| sibiricum | | | |
| Ganoderma lucidum | 1.27 | Raisin Tree Seed | 1.93 |
| Trametes versicolor | 1.31 | pericarpium citri | 2.19 |
| | | reticulatae | |
| mulberry leaf | 2.68 | the root bark of the | 2.68 |
| | | peony tree | |
| ginkgo leaf | 2.03 | Sedum | 2.17 |
| | | sarmentosum | |
| Lilium brownii | 1.68 | gardenia | 2.59 |
| Bear bile | 1.87 | Chinese wolfberry | 2.38 |
| Pig bile | 1.54 | White peony | 1.65 |
| Yinchen | 1.56 | Ligusticum | 1.51 |
| | | wallichii | |
| Fructus Aurantii | 1.68 | peach kernel | 1.29 |
| licorice | 1.44 | rheum officinale | 1.80 |
| Polygonum | 1.48 | chrysanthemum | 1.52 |
| cuspidatum | | | |
| Sophora japonica | 2.12 | serrate rabdosia | 2.34 |
| | | herb | |
| Hedyotis diffusa | 2.14 | radix bupleuri | 1.49 |
| the root of fangji | 1.68 | Poria cocos | 1.27 |
| lalang grass rhizome | 1.92 | Sculellaria barbata | 1.74 |

Although the invention has been described in detail with reference to preferred embodiments, variations and modifications exist within the scope and spirit of the invention as described and defined in the following claims.

What is claimed is:

1. A method for treating a nonalcoholic fatty liver disease (NAFLD), the method comprising administering to a subject an effective amount of a composition comprising at least one reagent promoting a tripartite motif-containing protein 31 (TRIM31) expression or a degradation of an rhomboid 5 homolog 2 (RHBDF2) protein.

2. The method of claim 1, wherein the NAFLD comprises liver lipid metabolism disorders and non-alcoholic steatohepatitis (NASH).

3. The method of claim 1, wherein the TRIM31 comprises the amino acid sequence shown in SEQ ID NO: 1 or SEQ ID NO: 2, and the nucleic acid sequence encoding the TRIM31 is shown in SEQ ID NO: 5 or SEQ ID NO: 6.

4. The method of claim 1, wherein the RHBDF2 comprises the amino acid sequence shown in SEQ ID NO: 3 or SEQ ID NO: 4, and the nucleic acid sequence encoding the RHBDF2 is shown in SEQ ID NO: 7 or SEQ ID NO: 8.

5. The method of claim 1, wherein the degradation of the RHBDF2 is promoted by a reagent linking E3 ligase to the RHBDF2.

6. The method of claim 1, wherein the degradation of the RHBDF2 is promoted by K48 ubiquitin linkage based on an interaction between TRIM31 and the RHBDF2.

7. The method of claim 5, wherein the E3 ligase is one or more of TRIM31, CRBN, VHL, TRIM26, and TRIM38.

8. The method of claim 1, wherein the at least one reagent comprises a recombinant lentiviral vector or adeno-associated virual vector packaging Trim31 with the nucleic acid sequence shown in SEQ ID NO: 5 or SEQ ID NO: 6.

9. The method of claim 1, wherein the at least one reagent also comprises one or more of the following substances:
(1) alkaloid compounds or their compositions, including magnoflorine, peganine, serpentine, leonurine, tetrandrine, orixine, peimine, colchicine, piperine, rhynchophylline, camptothecin, tetrahydropalmatine, jateorhizine, chelidonine, lycorine, acronycine, sophocarpidine, oxymatrine;
(2) flavonoids or their compositions, including quercetin, dihydroquercetin, luteolin, morin, Mulberrin, apigenin, diosmetin, chrysin, tricin, Puerarin, anthocyanin, proanthocyanidins, daidzein, baicalein, scutellarein, icariin, wogonin, cyanidin, silymarin, silibinin, genistein, galangin, kaempferol, fisetin, isorhamnetin, myricetin, hesperetin, naringenin, liquiritigenin, catechin, epicatechin, epigallocatechin, delphinidin, pelargonidin, breviscapine, afzelechin, rutin, luteolin-7-O-β-D-glucoside, baicalin, apiin, juglanin;
(3) polyphenolic compounds or their compositions, including chlorogenic acid, cynarin, cynarascoloside C, cynaropicrin, tea polyphenols, resveratrol, punicosides, curcumin, ferulic acid;
(4) amine compounds or their compositions, including penicillamine, pomadomide, lenalidomide, thalidomide;
(5) extracts of traditional Chinese medicine, including Astragalus membranaceus, Codonopsis pilosula, Salvia miltiorrhiza, Angelica sinensis, Chinese yam, pseudo-ginseng, Schisandra chinensis, Polygonatum sibiricum, Ganoderma lucidum, Trametes versicolor, artichoke, Thistle, milk thistle, Pueraria lobate, Coptis chinensis, Phellodendron amurense, turmeric, Honeysuckle, Raisin Tree Seed, pericarpium citri reticulatae, mulberry leaf, gingko leaf, lily, bear bile, pig bile, Yinchen, Fructus aurantii, Licorice, Polygonum cuspidatum, Sophora japonica, the root bark of the peony tree, Sedum sarmentosum, gardenia, Chinese Wolfberry, White Peony, Ligusticum wallichii, peach kernel, rheum officinale, chrysanthemum, serrate rabdosia herb, Hedyotis diffusa, the root of fangji, white grass root, Sculellaria barbata, radix bupleuri, Poria cocos;
(6) other compounds or their compositions, including Glycyrrhizinic acid, Glycyrrhizic acid monoammonium salt, adenosine, ursodeoxycholic acid, tauroursodeoxycholic acid, chenodeoxycholic acid, taurochenodeoxycholic acid, dicyclol, bifendate, sorafenib, regorafenib, levatinib, betacyanin, metformin, carminic acid.
